(19) United States
(12) Patent Application Publication
(10) Pub. No.: US 2018/0243550 A1
(43) Pub. Date: Aug. 30, 2018

(54) METHODS AND DEVICES FOR TISSUE TREATMENT

(71) Applicant: Vomaris Innovations, Inc., Tempe, AZ (US)

(72) Inventors: Troy Paluszcyk, Tempe, AZ (US); Wendell King, Pillager, MN (US)

(21) Appl. No.: 15/754,556

(22) PCT Filed: Aug. 25, 2016
(86) PCT No.: PCT/US16/48605

§ 371 Date: Feb. 22, 2018

Related U.S. Application Data
(60) Provisional application No. 62/210,337, filed on Aug. 26, 2015.
(60) Provisional application No. 62/246,723, filed on Oct. 27, 2015.

(51) Int. Cl. A61N 1/04; A61M 37/00; A61N 1/30

(52) U.S. Cl. CPC A61N 1/0496; A61N 1/0468; A61N 1/0476; A61N 1/328; A61M 37/00; A61N 1/303; A61M 2037/0007; A61N 1/0484

(57) ABSTRACT

Described are dehydrated bioelectric treatment devices including a hydrogel that when hydrated possess the properties of a device without the hydrogel.

### FIELD

Biologic tissues and cells are affected by electrical stimulus. Accordingly, apparatus and techniques for applying electric stimulus to biological tissue and cells have been developed to address a number of medical issues. The present specification relates to bioelectric devices and methods of manufacture and use thereof.

### SUMMARY

Embodiments disclosed herein include systems, devices, and methods for treating tissues. These tissues may have sustained injury and/or wounds (including surgical incisions), or could benefit from treatment for skin-related issues (for example, acne, rosacea, rash, or the like), or could benefit from treatment to minimize risk of injury (for example, muscle damage). Disclosed systems, devices, and methods can comprise a multi-array matrix of biocompatible microcells and provide a treatment site with a localized voltage and/or micro-current. Disclosed systems and devices can retain the ability to produce voltage and/or micro-current at a treatment site for a longer period of time than conventional devices, for example through the use of a hydrogel. For example, in an embodiment the system or device comprises a dehydrated hydrogel, which can provide a conductive environment upon re-hydration or reconstitution. Further, in certain embodiments the hydrogel helps to maintain a moist, conductive environment.

Hydrogels generally consist of a hydrophilic polymer combined with water to form a gel. In some embodiments, when all of the water is removed from the hydrogel, all that remains is a thin dry film of particles and crystals. When a bioelectric device, for example a multi-array matrix of biocompatible microcells on a backing sheet or base layer, is coated with a layer of hydrogel and allowed to dry (for example, at room temperature), the resulting dehydrated sheet has handling properties very similar to the device without the hydrogel present.

Disclosed methods, systems and devices can include a hydrogel that is not applied at the point of use. For example, in embodiments the hydrogel can be applied prior to use rendering it easy to use, clean, and convenient. For example, embodiments utilize water as a hydration element. Therefore, a user doesn't need to procure and apply a secondary, hard to find, wound hydrogel to be provided the benefit of extended hydration/battery activation. This can be particularly beneficial for non-hospital/surgical settings such as the battlefield or other “crisis” environments, over-the-counter uses, etc.

Methods, systems and devices disclosed herein can provide timed release of active agents. In embodiments, the disclosed hydrogels have the ability to sense changes in pH, temperature, or the concentration of a metabolite, and release their associated drug or active agent as result of such a change.

Disclosed embodiments comprise methods, systems, and devices. These methods, systems, and devices can include a dehydrated composition associated with or attached to or dried to or bonded to a base sheet or substrate. The dehydrated composition can include an array of microcells and a hydrogel. In some embodiments, when rehydrated, the dehydrated composition can provide a low level micro-current of between about 1 and about 400 micro-amperes. In other embodiments, the low level micro-current can be between about 1 and about 200 micro-amperes.

The base sheet or substrate can include an adhesive to hold the base sheet against the treatment site or can be provided or configured as a bandage. In disclosed embodiments, the hydrogel can be provided as a coating of hydrogel, for example on top of the array of microcells on a base layer or substrate. In various embodiments, the hydrogel can be provided on or in the treatment systems and devices for use at a concentration of, for example, about 0.1 g/in², 0.2 g/in², 0.3 g/in², 0.4 g/in², 0.5 g/in², 0.6 g/in², 0.7 g/in², 0.8 g/in², 0.9 g/in², 1.0 g/in², 1.1 g/in², 1.2 g/in², 1.3 g/in², 1.4 g/in², 1.5 g/in², 1.6 g/in², 1.7 g/in², 1.8 g/in², 1.9 g/in², 2.0 g/in², 3.0 g/in², 4.0 g/in², 5.0 g/in², 6.0 g/in², or 7.0 g/in² of the treatment device.

Other embodiments provide methods of treating the skin, for example, a wound, or a muscle. Embodiments disclosed herein include treatment of a muscle or muscle group (for example a muscle group surrounding a joint), either before, during, or after athletic activity or exercise. For example, a method of treatment disclosed herein can comprise applying an embodiment disclosed herein to the skin, or a muscle or muscle group.

Disclosed methods can include the steps of hydrating a composition including an array of microcells and a dehydrated hydrogel, thereby providing a hydrated composition, and applying the hydrated composition to the treatment site.

Embodiments disclosed herein can be used to treat irregular surfaces of the body, including the face, the shoulder, the elbow, the wrist, the finger joints, the hip, the knee, the ankle, the toe joints, etc. Additional embodiments disclosed herein can be used in areas where tissue is prone to movement, for example the eyelid, the ear, the lips, the nose, the shoulders, the back, etc.

Still other embodiments provide methods for forming systems and devices capable of providing a low level micro-current to a treatment area. Disclosed methods can comprise applying a hydrogel to an array of microcells associated with or attached or dried to or bonded to a base sheet and dehydrating the hydrogel. Disclosed methods can comprise applying a hydrogel comprising an array of microcells on to a base sheet and dehydrating the hydrogel. In embodiments, dehydrating the hydrogel preserves the low level micro-current producing potential of the device until the hydrogel is rehydrated. In some embodiments of the method, the hydrogel is applied as a coating. When the dehydrated system is exposed to water, saline, or other hydrating liquid, the rehydration produces a hydrated sheet very similar to the initial hydrated multi-array matrix of biocompatible microcells coated with a hydrogel on a backing sheet. The rehydrated sheet can perform like a bioelectric device without a hydrogel and produces a similar voltage at the site of treatment.

Disclosed embodiments can activate enzymes, increase glucose uptake, drive redox signaling, increase H₂O₂ production, increase cellular protein sulfhydryl levels, and increase (IGF)-1 R phosphorylation. Embodiments can also up-regulate integrin production and accumulation in treatment areas.

### BRIEF DESCRIPTION OF THE DRAWINGS

FIG. 1 is a detailed plan view of a base layer comprising a multi-array matrix of an embodiment disclosed herein. In an embodiment a hydrogel is applied to the base layer comprising the matrix, then dehydrated to dry the system. The system can then be re-hydrated prior to use.

FIG. 2 is a detailed plan view of a base layer comprising a pattern of applied electrical conductors in accordance with an embodiment disclosed herein.

FIG. 3 is an embodiment using the applied pattern of FIG. 2.

FIG. 4 is a cross-section of FIG. 3 through line 3-3.

FIG. 5 is a detailed plan view of an alternate embodiment disclosed herein which includes fine lines of conductive metal solution connecting electrodes upon the base layer.

FIG. 6 is a detailed plan view of another alternate embodiment having a line pattern and dot pattern.

FIG. 7 is a detailed plan view of yet another alternate embodiment having two line patterns.

FIGS. 8A-8E depict alternate embodiments showing the location of discontinuous regions as well as anchor regions of the system.

FIGS. 9A-9B depict alternate embodiments showing a garment comprising a multi-array matrix of biocompatible microcells.

FIGS. 10A-10B depict a “universal” embodiment for use on multiple areas of the body.

FIG. 11 depicts a detailed plan view of a substrate layer electrode pattern disclosed herein.

FIG. 12 depicts a detailed plan view of a substrate layer electrode pattern disclosed herein.

FIG. 13 depicts a detailed plan view of a substrate layer electrode pattern disclosed herein.

FIG. 14 depicts a graphical representation of a bioelectric hydrogel according to one or more embodiments disclosed herein; the hydrogel can be applied to a base layer then dehydrated.

FIG. 15 depicts a three-dimensional representation of a bioelectric hydrogel according to one or more embodiments; the hydrogel can be applied to a base layer then dehydrated.

### DETAILED DESCRIPTION

Described herein are systems, devices, and methods for treating tissues, for example organs such as skin or muscles, including skin conditions, wounds, and the like. These systems include bioelectric tissue care devices that comprise a multi-array matrix of biocompatible microcells and provide a treatment site with a localized voltage and/or amperage.

Embodiments disclosed herein comprise methods, systems and devices that can provide a low level electric field (LLEF) to a tissue or organism (thus a “LLEF system”) or, when brought into contact with an electrically conducting material, can provide a low level electric micro-current (LLEC) to a tissue or organism (thus a “LLEC system”). Thus, in embodiments a LLEC system is a LLEF system that is in contact with an electrically conducting material, for example a liquid material. In certain embodiments, the micro-current or electric field can be modulated, for example, to alter the duration, size, shape, field depth, duration, current, polarity, or voltage of the system. For example, it can be desirable to employ an electric field of greater strength or depth in an area to achieve optimal treatment. In embodiments the watt-density of the system can be modulated. Embodiments can comprise a gel, for example a hydrogel.

### Definitions

“Activation agent” as used herein means a composition useful for maintaining a moist environment within and about the skin. Activation agents can be in the form of gels (for example a hydrogel) or liquids. Activation agents can be conductive. Activation gels can also be antibacterial. In one embodiment, an activation agent can be a liquid such as sweat or topical substance such as petroleum jelly (for example with a conductive component added).

“Affixing” as used herein can mean contacting a patient or tissue with a device or system disclosed herein. In embodiments “affixing” can comprise the use of straps, elastic, etc.

“Antimicrobial agent” as used herein refers to an agent that kills or inhibits the growth of microorganisms. One type of antimicrobial agent can be an antibacterial agent. “Antibacterial agent” or “antibacterial” as used herein refers to an agent that interferes with the growth and reproduction of bacteria. Antibacterial agents are used to disinfect surfaces and eliminate potentially harmful bacteria. Unlike antibiotics, they are not used as medicines for humans or animals, but are found in products such as soaps, detergents, health and skincare products and household cleaners. Antibacterial agents may be divided into two groups according to their speed of action and residue production: The first group contains those that act rapidly to destroy bacteria, but quickly disappear (by evaporation or breakdown) and leave no active residue behind (referred to as non-residue-producing). Examples of this type are the alcohols, chlorine, peroxides, and aldehydes. The second group consists mostly of newer compounds that leave long-acting residues on the surface to be disinfected and thus have a prolonged action (referred to as residue-producing). Common examples of this group are triclosan, triclocarban, and benzalkonium chloride. Another type of antimicrobial agent can be an anti-fungal agent that can be used with the devices described herein.

“Applied” or “apply” as used herein refers to contacting a surface with a conductive material, for example printing, painting, or spraying a conductive ink on a surface. Alternatively, “applying” can mean contacting a patient or tissue or organism with a device or system disclosed herein.

“Conductive material” as used herein refers to an object or type of material which permits the flow of electric charges. Conductive materials can comprise solids such as metals or carbon, or liquids such as conductive metal solutions and conductive gels. Conductive materials can be applied to form at least one matrix. Conductive liquids can dry, cure, or harden after application to form a solid material.

“Discontinuous region” as used herein refers to a “void” in a material such as a hole, slot, or the like. The term can mean any void in the material though typically the void is of a regular shape. A void in the material can be entirely within the perimeter of a material or it can extend to the perimeter of a material.

“Dots” as used herein refers to discrete deposits of dissimilar reservoirs that can function as at least one battery cell. The term can refer to a deposit of any suitable size or shape, such as squares, circles, triangles, lines, etc. The term can be used synonymously with “electrodes,” “microcells,” “microspheres,” etc. “Microspheres” refers to are small spherical particles, with diameters in the micrometer range (typically 1 μm to 3000 μm (3 mm)). Microspheres are sometimes referred to as microparticles. Microspheres can be manufactured from various natural and synthetic materials. The term can be used synonymously with “micro-balloons,” “beads,” “particles,” etc.

“Electrode” refers to similar or dissimilar conductive materials. In embodiments utilizing an external power source the electrodes can comprise similar conductive materials. In embodiments that do not use an external power source, the electrodes can comprise dissimilar conductive materials that can define an anode and a cathode.

“Expandable” as used herein refers to the ability to stretch while retaining structural integrity and not tearing. The term can refer to solid regions as well as discontinuous or void regions; solid regions as well as void regions can stretch or expand.

“Matrix” or “matrices” or “array” or “arrays” as used herein refer to a pattern or patterns, such as those formed by electrodes on a surface, such as a fabric or a fiber, or the like. Matrices can also comprise a pattern or patterns within a solid or liquid material or a three dimensional object. Matrices can be designed to vary the electric field or electric current or microcurrent generated. For example, the strength and shape of the field or current or microcurrent can be altered, or the matrices can be designed to produce an electric field(s) or current or microcurrent of a desired strength or shape. “Matrices” can also refer to the random distribution of electrodes in a gel, such as a hydrogel.

“Sheets” as used herein refer to substrate, typically in bulk quantities. As such, “sheets” can refer to a continuous roll or unit of substrate.

“Stretchable” as used herein refers to the ability of embodiments that stretch without losing their structural integrity. That is, embodiments can stretch to accommodate irregular skin surfaces or surfaces wherein one portion of the surface can move relative to another portion.

“Treatment” as used herein can include the use of disclosed embodiments on tissue to prevent, reduce, or repair damage. Treatment can also include the use of disclosed embodiments on the skin, eyes, etc. Treatment can include use on an injury, for example a wound.

“Viscosity” as used herein refers to a measurement of a fluid's resistance to gradual deformation by shear stress or tensile stress. That is, embodiments can accommodate multiple viscosity variations without losing structural integrity, wherein one embodiment can be a liquid or a solid material.

LLEC/LLEF Systems, Devices, and Methods of Manufacture

In embodiments, disclosed methods, systems, and devices can retain their ability to provide localized voltage and/or amperage at a treatment site for a sustained period of time. In embodiments, this sustained period of time can be achieved by including a hydrogel in or with the multi-array matrix of biocompatible microcells and dehydrating the hydrogel. Once dehydrated, the device can be stored without losing its ability to later deliver a localized voltage and/or amperage. The localized voltage and/or amperage can be triggered or activated by rehydrating the hydrogel as described herein.

In embodiments, disclosed systems and devices can, in their dehydrated state, retain their ability to provide, upon re-hydration, localized voltage and/or amperage (“shelf” stability) for more than about 1 week, more that about 2 weeks, more than about 3 weeks, more than about 1 month, more than about 2 months, more than about 3 months, more than about 4 months, more than about 5 months, more than about 6 months, more than about 7 months, more than about 8 months, more than about 9 months, more than about 10 months, more than about 11 months, more than about 1 year, more than about 2 years, more than about 3 years, more than about 4 years, more than about 5 years, more than about 6 years, more than about 7 years, more than about 8 years, more than about 9 years, more than about 10 years, or more.

In some embodiments, the systems and devices described herein can include a backing sheet or base layer or substrate, a multi-array matrix of biocompatible microcells and a hydrogel. In other embodiments, a combination of two or more hydrogels can be used. The hydrogel or hydrogel(s) can be dehydrated to allow for future voltage and/or current/amperage delivery of a device once rehydrated. In certain embodiments, the systems and devices are configured to be hydrated at the time of use, for example, by including a removable protective layer over the device's active surface. The active surface can be the surface of the device that will touch or otherwise interface with the treatment location.

In embodiments, the herein-described methods, systems, and devices provide a multi-array matrix of biocompatible microcells coated or otherwise impregnated with a hydrogel which can then be dried to remove the water in the hydrogel. Hydrogels generally include a hydrophilic polymer combined with water to form a gel. In some embodiments, when all of the water is removed from the hydrogel, all that remains is a thin dry film of particles and crystals. When a bioelectric device, for example a multi-array matrix of biocompatible microcells on a backing sheet, is coated with a layer of hydrogel and allowed to dry at room temperature, the resulting dehydrated sheet has handling properties substantially similar to the device without the hydrogel present. Substantially similar includes devices that retain more than about 80%, more than about 85%, more than about 90%, more than about 95%, or more than about 99% of the function of the device without a hydrogel. In some embodiments, a device with a hydrogel retains all the function of a device without a hydrogel.

The methods, systems, and devices described herein can comprise a multi-array matrix of biocompatible microcells that can produce a localized treatment voltage or microcurrent or both at a treatment site. In some embodiments, the voltage can be a low level electric field (LLEF). This electric filed can be delivered to a tissue or organism (thus a “LLEF system”) or, when brought into contact with an electrically conducting material, can provide a low level electric micro-current (LLEC) to a tissue or organism (thus a “LLEC system”). Thus, in embodiments a LLEC system is a LLEF system that is in contact with an electrically conducting material, for example a liquid material. In certain embodiments, the micro-current or electric field can be modulated, for example, to alter the duration, size, shape, field depth, duration, current, polarity, or voltage of the system. In embodiments, the field is very short, for example in the range of physiologic electric fields. In some embodiments, the direction of the electric field produced by devices disclosed herein is omnidirectional over the surface of the wound and more in line with the physiologic electric fields.

In some embodiments, the multi-array matrix of biocompatible microcells can comprise a first array comprising a pattern of microcells formed of a conductive material and a second array comprising a pattern of microcells formed from a second conductive material. The first conductive material can be formed from, for example, a first conductive solution and the second conductive material can be formed from, for example, a second conductive solution. The first and/or second conductive solutions can include a metal species such as a metal species capable of defining at least one voltaic cell for spontaneously generating at least one electrical current with the metal species of the first array when said first and second arrays are introduced to an electrolytic solution and said first and second arrays are not in physical contact with each other. Certain embodiments utilize an external power source such as AC or DC power, or pulsed RF, or pulsed current, such as high voltage pulsed current. In one embodiment, the electrical energy is derived from the dissimilar metals creating a battery at each electrode/electrode interface, whereas those embodiments with an external power source can employ conductive electrodes in a spaced configuration to predetermine the electric field shape and strength.

In certain embodiments, for example treatment methods, it can be preferable to utilize AC or DC current. For example, embodiments disclosed herein can employ phased array, pulsed, square wave, sinusoidal, or other wave forms, combinations, or the like. Certain embodiments utilize a controller to produce and control power production and/or distribution to the device.

Embodiments of the LLEC or LLEF methods, systems, and devices disclosed herein can comprise electrodes or microcells. Electrodes or microcells can comprise discrete deposits of dissimilar reservoirs that can function as at least one battery cell. The deposits can be of any suitable size or shape, such as squares, circles, triangles, lines, etc. In some embodiments, “dots” can be used synonymously with, “microcells,” and the like. Each electrode or microcell can be or comprise a conductive material, for example, a metal. In embodiments, the electrodes or microcells can comprise any electrically-conductive material, for example, an electrically conductive hydrogel, metals, electrolytes, superconductors, semiconductors, plasmas, and nonmetallic conductors such as graphite and conductive polymers. Electrically conductive metals can include, for example, silver, copper, gold, aluminum, molybdenum, zinc, lithium, tungsten, brass, carbon, nickel, iron, palladium, platinum, tin, bronze, carbon steel, lead, titanium, stainless steel, mercury, Fe/Cr alloys, and the like. The electrode can be coated or plated with a different metal such as aluminum, gold, platinum or silver.

In embodiments, the hydrated gel can be associated with a substrate by any suitable means. For example, in an embodiment, a wet and/or amorphous gel can be applied to a substrate then dehydrated. The embodiment is activated upon rehydration. In an alternate embodiment, a specially formulated wet/amorphous gel that lacks conductive properties/ions (that remains in that state until water is applied) is applied to a substrate, then dehydrated. The embodiment is activated upon rehydration.

Another embodiment comprises applying to a substrate a “dry” gel that is made up only of the volatiles of an amorphous gel. The embodiment is activated upon rehydration. Another embodiment comprises applying a hydrogel sheet/film (hot melt extrusion) that does not produce a microcurrent until a liquid is applied and absorbed into the sheet/film.

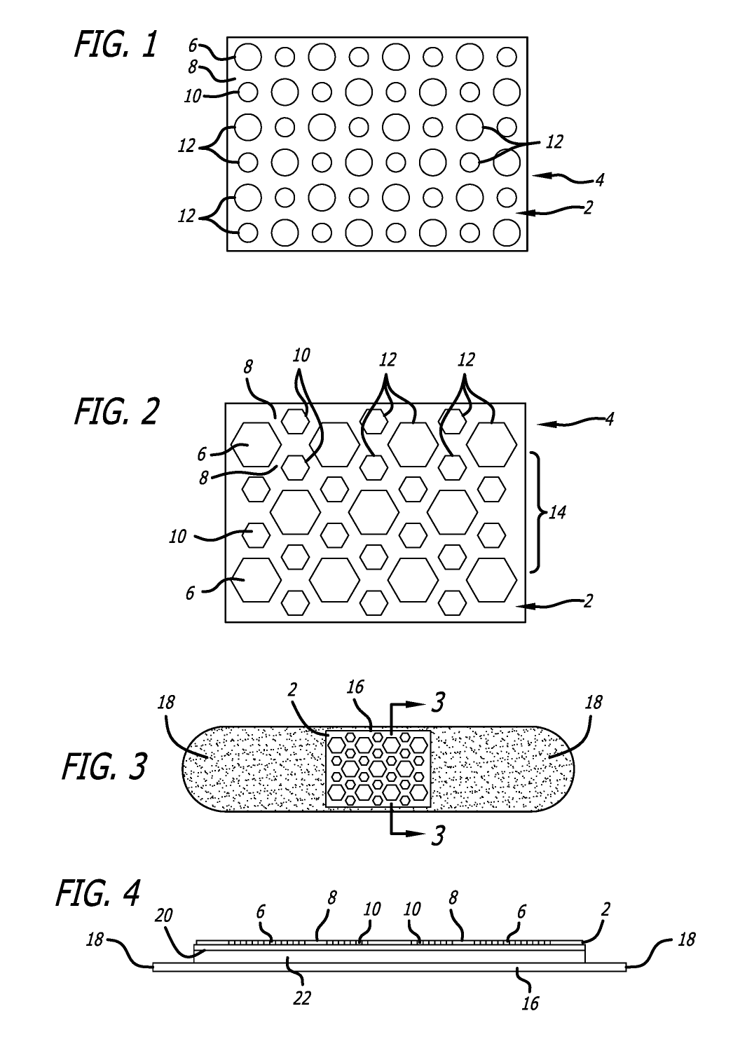

Turning to the figures, in FIG. 1, the dissimilar first electrode 6 and second electrode 10 are applied onto base layer or substrate 2 of an article 4, for example a fabric. In an embodiment a primary surface is a surface of a LLEC or LLEF system that comes into direct contact with an area to be treated, for example a skin surface.

In various embodiments the difference of the standard potentials of the electrodes or dots or reservoirs can be in a range from about 0.05 V to approximately about 5.0 V. For example, the standard potential can be about 0.05 V, about 0.06 V, about 0.07 V, about 0.08 V, about 0.09 V, about 0.1 V, about 0.2 V, about 0.3 V, about 0.4 V, about 0.5 V, about 0.6 V, about 0.7 V, about 0.8 V, about 0.9 V, about 1.0 V, about 1.1 V, about 1.2 V, about 1.3 V, about 1.4 V, about 1.5 V, about 1.6 V, about 1.7 V, about 1.8 V, about 1.9 V, about 2.0 V, about 2.1 V, about 2.2 V, about 2.3 V, about 2.4 V, about 2.5 V, about 2.6 V, about 2.7 V, about 2.8 V, about 2.9 V, about 3.0 V, about 3.1 V, about 3.2 V, about 3.3 V, about 3.4 V, about 3.5 V, about 3.6 V, about 3.7 V, about 3.8 V, about 3.9 V, about 4.0 V, about 4.1 V, about 4.2 V, about 4.3 V, about 4.4 V, about 4.5 V, about 4.6 V, about 4.7 V, about 4.8 V, about 4.9 V, about 5.0 V, about 5.1 V, about 5.2 V, about 5.3 V, about 5.4 V, about 5.5 V, about 5.6 V, about 5.7 V, about 5.8 V, about 5.9 V, about 6.0 V, about 6.1 V, about 6.2 V, about 6.3 V, about 6.4 V, about 6.5 V, about 6.6 V, about 6.7 V, about 6.8 V, about 6.9 V, about 7.0 V, about 7.1 V, about 7.2 V, about 7.3 V, about 7.4 V, about 7.5 V, about 7.6 V, about 7.7 V, about 7.8 V, about 7.9 V, about 8.0 V, about 8.1 V, about 8.2 V, about 8.3 V, about 8.4 V, about 8.5 V, about 8.6 V, about 8.7 V, about 8.8 V, about 8.9 V, about 9.0 V, or the like.

In other embodiments the difference of the standard potentials of electrodes or dots or reservoirs can be at least 0.05 V, at least 0.06 V, at least 0.07 V, at least 0.08 V, at least 0.09 V, at least 0.1 V, at least 0.2 V, at least 0.3 V, at least 0.4 V, at least 0.5 V, at least 0.6 V, at least 0.7 V, at least 0.8 V, at least 0.9 V, at least 1.0 V, at least 1.1 V, at least 1.2 V, at least 1.3 V, at least 1.4 V, at least 1.5 V, at least 1.6 V, at least 1.7 V, at least 1.8 V, at least 1.9 V, at least 2.0 V, at least 2.1 V, at least 2.2 V, at least 2.3 V, at least 2.4 V, at least 2.5 V, at least 2.6 V, at least 2.7 V, at least 2.8 V, at least 2.9 V, at least 3.0 V, at least 3.1 V, at least 3.2 V, at least 3.3 V, at least 3.4 V, at least 3.5 V, at least 3.6 V, at least 3.7 V, at least 3.8 V, at least 3.9 V, at least 4.0 V, at least 4.1 V, at least 4.2 V, at least 4.3 V, at least 4.4 V, at least 4.5 V, at least 4.6 V, at least 4.7 V, at least 4.8 V, at least 4.9 V, at least 5.0 V, or the like, once the device or system is rehydrated for use from a dehydrated state.

In still other embodiments, the difference of the standard potentials of electrodes or dots or reservoirs can be less than 0.05 V, less than 0.06 V, less than 0.07 V, less than 0.08 V, less than 0.09 V, less than 0.1 V, less than 0.2 V, less than 0.3 V, less than 0.4 V, less than 0.5 V, less than 0.6 V, less than 0.7 V, less than 0.8 V, less than 0.9 V, less than 1.0 V, less than 1.1 V, less than 1.2 V, less than 1.3 V, less than 1.4 V, less than 1.5 V, less than 1.6 V, less than 1.7 V, less than 1.8 V, less than 1.9 V, less than 2.0 V, less than 2.1 V, less than 2.2 V, less than 2.3 V, less than 2.4 V, less than 2.5 V, less than 2.6 V, less than 2.7 V, less than 2.8 V, less than 2.9 V, less than 3.0 V, less than 3.1 V, less than 3.2 V, less than 3.3 V, less than 3.4 V, less than 3.5 V, less than 3.6 V, less than 3.7 V, less than 3.8 V, less than 3.9 V, less than 4.0 V, less than 4.1 V, less than 4.2 V, less than 4.3 V, less than 4.4 V, less than 4.5 V, less than 4.6 V, less e than 4.7 V, less than 4.8 V, less than 4.9 V, less than 5.0 V, or the like, once the device or system is rehydrated for use from a dehydrated state.

In embodiments, systems and devices disclosed herein can produce a low level electric current of between for example about 1 and about 200 micro-amperes, between about 10 and about 190 micro-amperes, between about 20 and about 180 micro-amperes, between about 30 and about 170 micro-amperes, between about 40 and about 160 micro-amperes, between about 50 and about 150 micro-amperes, between about 60 and about 140 micro-amperes, between about 70 and about 130 micro-amperes, between about 80 and about 120 micro-amperes, between about 90 and about 100 micro-amperes, between about 100 and about 150 micro-amperes, between about 150 and about 200 micro-amperes, between about 200 and about 250 micro-amperes, between about 250 and about 300 micro-amperes, between about 300 and about 350 micro-amperes, between about 350 and about 400 micro-amperes, between about 400 and about 450 micro-amperes, between about 450 and about 500 micro-amperes, between about 500 and about 550 micro-amperes, between about 550 and about 600 micro-amperes, between about 600 and about 650 micro-amperes, between about 650 and about 700 micro-amperes, between about 700 and about 750 micro-amperes, between about 750 and about 800 micro-amperes, between about 800 and about 850 micro-amperes, between about 850 and about 900 micro-amperes, between about 900 and about 950 micro-amperes, between about 950 and about 1000 micro-amperes (1 milli-amp [mA]), between about 1.0 and about 1.1 mA, between about 1.1 and about 1.2 mA, between about 1.2 and about 1.3 mA, between about 1.3 and about 1.4 mA, between about 1.4 and about 1.5 mA, between about 1.5 and about 1.6 mA, between about 1.6 and about 1.7 mA, between about 1.7 and about 1.8 mA, between about 1.8 and about 1.9 mA, between about 1.9 and about 2.0 mA, between about 2.0 and about 2.1 mA, between about 2.1 and about 2.2 mA, between about 2.2 and about 2.3 mA, between about 2.3 and about 2.4 mA, between about 2.4 and about 2.5 mA, between about 2.5 and about 2.6 mA, between about 2.6 and about 2.7 mA, between about 2.7 and about 2.8 mA, between about 2.8 and about 2.9 mA, between about 2.9 and about 3.0 mA, between about 3.0 and about 3.1 mA, between about 3.1 and about 3.2 mA, between about 3.2 and about 3.3 mA, between about 3.3 and about 3.4 mA, between about 3.4 and about 3.5 mA, between about 3.5 and about 3.6 mA, between about 3.6 and about 3.7 mA, between about 3.7 and about 3.8 mA, between about 3.8 and about 3.9 mA, between about 3.9 and about 4.0 mA, between about 4.0 and about 4.1 mA, between about 4.1 and about 4.2 mA, between about 4.2 and about 4.3 mA, between about 4.3 and about 4.4 mA, between about 4.4 and about 4.5 mA, between about 4.5 and about 5.0 mA, between about 5.0 and about 5.5 mA, between about 5.5 and about 6.0 mA, between about 6.0 and about 6.5 mA, between about 6.5 and about 7.0 mA, between about 7.5 and about 8.0 mA, between about 8.0 and about 8.5 mA, between about 8.5 and about 9.0 mA, between about 9.0 and about 9.5 mA, between about 9.5 and about 10.0 mA, between about 10.0 and about 10.5 mA, between about 10.5 and about 11.0 mA, between about 11.0 and about 11.5 mA, between about 11.5 and about 12.0 mA, between about 12.0 and about 12.5 mA, between about 12.5 and about 13.0 mA, between about 13.0 and about 13.5 mA, between about 13.5 and about 14.0 mA, between about 14.0 and about 14.5 mA, between about 14.5 and about 15.0 mA, or the like.

In embodiments, systems and devices disclosed herein can produce a low level electric current of between for example about 1 and about 400 micro-amperes, between about 20 and about 380 micro-amperes, between about 40 and about 360 micro-amperes, between about 60 and about 340 micro-amperes, between about 80 and about 320 micro-amperes, between about 100 and about 300 micro-amperes, between about 120 and about 280 micro-amperes, between about 140 and about 260 micro-amperes, between about 160 and about 240 micro-amperes, between about 180 and about 220 micro-amperes, or the like.

In embodiments, systems and devices disclosed herein can produce a low level electric current of between for example about 1 micro-ampere and about 1 milli-ampere, between about 50 and about 800 micro-amperes, between about 200 and about 600 micro-amperes, between about 400 and about 500 micro-amperes, or the like.

In embodiments, systems and devices disclosed herein can produce a low level electric current of about 10 micro-amperes, about 20 micro-amperes, about 30 micro-amperes, about 40 micro-amperes, about 50 micro-amperes, about 60 micro-amperes, about 70 micro-amperes, about 80 micro-amperes, about 90 micro-amperes, about 100 micro-amperes, about 110 micro-amperes, about 120 micro-amperes, about 130 micro-amperes, about 140 micro-amperes, about 150 micro-amperes, about 160 micro-amperes, about 170 micro-amperes, about 180 micro-amperes, about 190 micro-amperes, about 200 micro-amperes, about 210 micro-amperes, about 220 micro-amperes, about 240 micro-amperes, about 260 micro-amperes, about 280 micro-amperes, about 300 micro-amperes, about 320 micro-amperes, about 340 micro-amperes, about 360 micro-amperes, about 380 micro-amperes, about 400 micro-amperes, about 450 micro-amperes, about 500 micro-amperes, about 550 micro-amperes, about 600 micro-amperes, about 650 micro-amperes, about 700 micro-amperes, about 750 micro-amperes, about 800 micro-amperes, about 850 micro-amperes, about 900 micro-amperes, about 950 micro-amperes, about 1 milli-ampere (mA), about 1.1 mA, about 1.2 mA, about 1.3 mA, about 1.4 mA, about 1.5 mA, about 1.6 mA, about 1.7 mA, about 1.8 mA, about 1.9 mA, about 2.0 mA, about 2.1 mA, about 2.2 mA, about 2.3 mA, about 2.4 mA, about 2.5 mA, about 2.6 mA, about 2.7 mA, about 2.8 mA, about 2.9 mA, about 3.0 mA, about 3.1 mA, about 3.2 mA, about 3.3 mA, about 3.4 mA, about 3.5 mA, about 3.6 mA, about 3.7 mA, about 3.8 mA, about 3.9 mA, about 4.0 mA, about 4.1 mA, about 4.2 mA, about 4.3 mA, about 4.4 mA, about 4.5 mA, about 4.6 mA, about 4.7 mA, about 4.8 mA, about 4.9 mA, about 5.0 mA, about 5.1 mA, about 5.2 mA, about 5.3 mA, about 5.4 mA, about 5.5 mA, about 5.6 mA, about 5.7 mA, about 5.8 mA, about 5.9 mA, about 6.0 mA, about 6.1 mA, about 4.2 mA, about 6.3 mA, about 6.4 mA, about 6.5 mA, about 6.6 mA, about 6.7 mA, about 6.8 mA, about 6.9 mA, about 7.0 mA, about 7.1 mA, about 7.2 mA, about 7.3 mA, about 7.4 mA, about 7.5 mA, about 7.6 mA, about 7.7 mA, about 7.8 mA, about 7.9 mA, about 8.0 mA, about 8.1 mA, about 8.2 mA, about 8.3 mA, about 8.4 mA, about 8.5 mA, about 8.6 mA, about 8.7 mA, about 8.8 mA, about 8.9 mA, about 9.0 mA, about 9.1 mA, about 9.2 mA, about 9.3 mA, about 9.4 mA, about 9.5 mA, about 9.6 mA, about 9.7 mA, about 9.8 mA, about 9.9 mA, about 10.0 mA, about 10.1 mA, about 10.2 mA, about 10.3 mA, about 10.4 mA, about 10.5 mA, about 10.6 mA, about 10.7 mA, about 10.8 mA, about 10.9 mA, about 11.0 mA, about 11.1 mA, about 11.2 mA, about 11.3 mA, about 11.4 mA, about 11.5 mA, about 11.6 mA, about 11.7 mA, about 11.8 mA, about 11.9 mA, about 12.0 mA, about 12.1 mA, about 12.2 mA, about 12.3 mA, about 12.4 mA, about 12.5 mA, about 12.6 mA, about 12.7 mA, about 12.8 mA, about 12.9 mA, about 13.0 mA, about 13.1 mA, about 13.2 mA, about 13.3 mA, about 13.4 mA, about 13.5 mA, about 13.6 mA, about 13.7 mA, about 13.8 mA, about 13.9 mA, about 14.0 mA, about 14.1 mA, about 14.2 mA, about 14.3 mA, about 14.4 mA, about 14.5 mA, about 14.6 mA, about 14.7 mA, about 14.8 mA, about 14.9 mA, about 15.0 mA, about 15.1 mA, about 15.2 mA, about 15.3 mA, about 15.4 mA, about 15.5 mA, about 15.6 mA, about 15.7 mA, about 15.8 mA, or the like.

In embodiments, the disclosed systems and devices can produce a low level electric current of not more than about 10 micro-amperes, or not more than about 20 micro-amperes, not more than about 30 micro-amperes, not more than about 40 micro-amperes, not more than about 50 micro-amperes, not more than about 60 micro-amperes, not more than about 70 micro-amperes, not more than about 80 micro-amperes, not more than about 90 micro-amperes, not more than about 100 micro-amperes, not more than about 110 micro-amperes, not more than about 120 micro-amperes, not more than about 130 micro-amperes, not more than about 140 micro-amperes, not more than about 150 micro-amperes, not more than about 160 micro-amperes, not more than about 170 micro-amperes, not more than about 180 micro-amperes, not more than about 190 micro-amperes, not more than about 200 micro-amperes, not more than about 210 micro-amperes, not more than about 220 micro-amperes, not more than about 230 micro-amperes, not more than about 240 micro-amperes, not more than about 250 micro-amperes, not more than about 260 micro-amperes, not more than about 270 micro-amperes, not more than about 280 micro-amperes, not more than about 290 micro-amperes, not more than about 300 micro-amperes, not more than about 310 micro-amperes, not more than about 320 micro-amperes, not more than about 340 micro-amperes, not more than about 360 micro-amperes, not more than about 380 micro-amperes, not more than about 400 micro-amperes, not more than about 420 micro-amperes, not more than about 440 micro-amperes, not more than about 460 micro-amperes, not more than about 480 micro-amperes, not more than about 500 micro-amperes, not more than about 520 micro-amperes, not more than about 540 micro-amperes, not more than about 560 micro-amperes, not more than about 580 micro-amperes, not more than about 600 micro-amperes, not more than about 620 micro-amperes, not more than about 640 micro-amperes, not more than about 660 micro-amperes, not more than about 680 micro-amperes, not more than about 700 micro-amperes, not more than about 720 micro-amperes, not more than about 740 micro-amperes, not more than about 760 micro-amperes, not more than about 780 micro-amperes, not more than about 800 micro-amperes, not more than about 820 micro-amperes, not more than about 840 micro-amperes, not more than about 860 micro-amperes, not more than about 880 micro-amperes, not more than about 900 micro-amperes, not more than about 920 micro-amperes, not more than about 940 micro-amperes, not more than about 960 micro-amperes, not more than about 980 micro-amperes, not more than about 1 milli-ampere (mA), not more than about 1.1 mA, not more than about 1.2 mA, not more than about 1.3 mA, not more than about 1.4 mA, not more than about 1.5 mA, not more than about 1.6 mA, not more than about 1.7 mA, not more than about 1.8 mA, not more than about 1.9 mA, not more than about 2.0 mA, not more than about 2.1 mA, not more than about 2.2 mA, not more than about 2.3 mA, not more than about 2.4 mA, not more than about 2.5 mA, not more than about 2.6 mA, not more than about 2.7 mA, not more than about 2.8 mA, not more than about 2.9 mA, not more than about 3.0 mA, not more than about 3.1 mA, not more than about 3.2 mA, not more than about 3.3 mA, not more than about 3.4 mA, not more than about 3.5 mA, not more than about 3.6 mA, not more than about 3.7 mA, not more than about 3.8 mA, not more than about 3.9 mA, not more than about 4.0 mA, not more than about 4.1 mA, not more than about 4.2 mA, not more than about 4.3 mA, not more than about 4.4 mA, not more than about 4.5 mA, not more than about 4.6 mA, not more than about 4.7 mA, not more than about 4.8 mA, not more than about 4.9 mA, not more than about 5.0 mA, not more than about 5.1 mA, not more than about 5.2 mA, not more than about 5.3 mA, not more than about 5.4 mA, not more than about 5.5 mA, not more than about 5.6 mA, not more than about 5.7 mA, not more than about 5.8 mA, not more than about 5.9 mA, not more than about 6.0 mA, not more than about 6.1 mA, not more than about 4.2 mA, not more than about 6.3 mA, not more than about 6.4 mA, not more than about 6.5 mA, not more than about 6.6 mA, not more than about 6.7 mA, not more than about 6.8 mA, not more than about 6.9 mA, not more than about 7.0 mA, not more than about 7.1 mA, not more than about 7.2 mA, not more than about 7.3 mA, not more than about 7.4 mA, not more than about 7.5 mA, not more than about 7.6 mA, not more than about 7.7 mA, not more than about 7.8 mA, not more than about 7.9 mA, not more than about 8.0 mA, not more than about 8.1 mA, not more than about 8.2 mA, not more than about 8.3 mA, not more than about 8.4 mA, not more than about 8.5 mA, not more than about 8.6 mA, not more than about 8.7 mA, not more than about 8.8 mA, not more than about 8.9 mA, not more than about 9.0 mA, not more than about 9.1 mA, not more than about 9.2 mA, not more than about 9.3 mA, not more than about 9.4 mA, not more than about 9.5 mA, not more than about 9.6 mA, not more than about 9.7 mA, not more than about 9.8 mA, not more than about 9.9 mA, not more than about 10.0 mA, not more than about 10.1 mA, not more than about 10.2 mA, not more than about 10.3 mA, not more than about 10.4 mA, not more than about 10.5 mA, not more than about 10.6 mA, not more than about 10.7 mA, not more than about 10.8 mA, not more than about 10.9 mA, not more than about 11.0 mA, not more than about 11.1 mA, not more than about 11.2 mA, not more than about 11.3 mA, not more than about 11.4 mA, not more than about 11.5 mA, not more than about 11.6 mA, not more than about 11.7 mA, not more than about 11.8 mA, not more than about 11.9 mA, not more than about 12.0 mA, not more than about 12.1 mA, not more than about 12.2 mA, not more than about 12.3 mA, not more than about 12.4 mA, not more than about 12.5 mA, not more than about 12.6 mA, not more than about 12.7 mA, not more than about 12.8 mA, not more than about 12.9 mA, not more than about 13.0 mA, not more than about 13.1 mA, not more than about 13.2 mA, not more than about 13.3 mA, not more than about 13.4 mA, not more than about 13.5 mA, not more than about 13.6 mA, not more than about 13.7 mA, not more than about 13.8 mA, not more than about 13.9 mA, not more than about 14.0 mA, not more than about 14.1 mA, not more than about 14.2 mA, not more than about 14.3 mA, not more than about 14.4 mA, not more than about 14.5 mA, not more than about 14.6 mA, not more than about 14.7 mA, not more than about 14.8 mA, not more than about 14.9 mA, not more than about 15.0 mA, not more than about 15.1 mA, not more than about 15.2 mA, not more than about 15.3 mA, not more than about 15.4 mA, not more than about 15.5 mA, not more than about 15.6 mA, not more than about 15.7 mA, not more than about 15.8 mA, and the like.

In embodiments, systems and devices disclosed herein can produce a low level electric current of not less than 10 micro-amperes, not less than 20 micro-amperes, not less than 30 micro-amperes, not less than 40 micro-amperes, not less than 50 micro-amperes, not less than 60 micro-amperes, not less than 70 micro-amperes, not less than 80 micro-amperes, not less than 90 micro-amperes, not less than 100 micro-amperes, not less than 110 micro-amperes, not less than 120 micro-amperes, not less than 130 micro-amperes, not less than 140 micro-amperes, not less than 150 micro-amperes, not less than 160 micro-amperes, not less than 170 micro-amperes, not less than 180 micro-amperes, not less than 190 micro-amperes, not less than 200 micro-amperes, not less than 210 micro-amperes, not less than 220 micro-amperes, not less than 230 micro-amperes, not less than 240 micro-amperes, not less than 250 micro-amperes, not less than 260 micro-amperes, not less than 270 micro-amperes, not less than 280 micro-amperes, not less than 290 micro-amperes, not less than 300 micro-amperes, not less than 310 micro-amperes, not less than 320 micro-amperes, not less than 330 micro-amperes, not less than 340 micro-amperes, not less than 350 micro-amperes, not less than 360 micro-amperes, not less than 370 micro-amperes, not less than 380 micro-amperes, not less than 390 micro-amperes, not less than 400 micro-amperes, not less than about 420 micro-amperes, not less than about 440 micro-amperes, not less than about 460 micro-amperes, not less than about 480 micro-amperes, not less than about 500 micro-amperes, not less than about 520 micro-amperes, not less than about 540 micro-amperes, not less than about 560 micro-amperes, not less than about 580 micro-amperes, not less than about 600 micro-amperes, not less than about 620 micro-amperes, not less than about 640 micro-amperes, not less than about 660 micro-amperes, not less than about 680 micro-amperes, not less than about 700 micro-amperes, not less than about 720 micro-amperes, not less than about 740 micro-amperes, not less than about 760 micro-amperes, not less than about 780 micro-amperes, not less than about 800 micro-amperes, not less than about 820 micro-amperes, not less than about 840 micro-amperes, not less than about 860 micro-amperes, not less than about 880 micro-amperes, not less than about 900 micro-amperes, not less than about 920 micro-amperes, not less than about 940 micro-amperes, not less than about 960 micro-amperes, not less than about 980 micro-amperes, not less than about 1 milli-ampere (mA), not less than about 1.1 mA, not less than about 1.2 mA, not less than about 1.3 mA, not less than about 1.4 mA, not less than about 1.5 mA, not less than about 1.6 mA, not less than about 1.7 mA, not less than about 1.8 mA, not less than about 1.9 mA, not less than about 2.0 mA, not less than about 2.1 mA, not less than about 2.2 mA, not less than about 2.3 mA, not less than about 2.4 mA, not less than about 2.5 mA, not less than about 2.6 mA, not less than about 2.7 mA, not less than about 2.8 mA, not less than about 2.9 mA, not less than about 3.0 mA, not less than about 3.1 mA, not less than about 3.2 mA, not less than about 3.3 mA, not less than about 3.4 mA, not less than about 3.5 mA, not less than about 3.6 mA, not less than about 3.7 mA, not less than about 3.8 mA, not less than about 3.9 mA, not less than about 4.0 mA, not less than about 4.1 mA, not less than about 4.2 mA, not less than about 4.3 mA, not less than about 4.4 mA, not less than about 4.5 mA, not less than about 4.6 mA, not less than about 4.7 mA, not less than about 4.8 mA, not less than about 4.9 mA, not less than about 5.0 mA, not less than about 5.1 mA, not less than about 5.2 mA, not less than about 5.3 mA, not less than about 5.4 mA, not less than about 5.5 mA, not less than about 5.6 mA, not less than about 5.7 mA, not less than about 5.8 mA, not less than about 5.9 mA, not less than about 6.0 mA, not less than about 6.1 mA, not less than about 4.2 mA, not less than about 6.3 mA, not less than about 6.4 mA, not less than about 6.5 mA, not less than about 6.6 mA, not less than about 6.7 mA, not less than about 6.8 mA, not less than about 6.9 mA, not less than about 7.0 mA, not less than about 7.1 mA, not less than about 7.2 mA, not less than about 7.3 mA, not less than about 7.4 mA, not less than about 7.5 mA, not less than about 7.6 mA, not less than about 7.7 mA, not less than about 7.8 mA, not less than about 7.9 mA, not less than about 8.0 mA, not less than about 8.1 mA, not less than about 8.2 mA, not less than about 8.3 mA, not less than about 8.4 mA, not less than about 8.5 mA, not less than about 8.6 mA, not less than about 8.7 mA, not less than about 8.8 mA, not less than about 8.9 mA, not less than about 9.0 mA, not less than about 9.1 mA, not less than about 9.2 mA, not less than about 9.3 mA, not less than about 9.4 mA, not less than about 9.5 mA, not less than about 9.6 mA, not less than about 9.7 mA, not less than about 9.8 mA, not less than about 9.9 mA, not less than about 10.0 mA, not less than about 10.1 mA, not less than about 10.2 mA, not less than about 10.3 mA, not less than about 10.4 mA, not less than about 10.5 mA, not less than about 10.6 mA, not less than about 10.7 mA, not less than about 10.8 mA, not less than about 10.9 mA, not less than about 11.0 mA, not less than about 11.1 mA, not less than about 11.2 mA, not less than about 11.3 mA, not less than about 11.4 mA, not less than about 11.5 mA, not less than about 11.6 mA, not less than about 11.7 mA, not less than about 11.8 mA, not less than about 11.9 mA, not less than about 12.0 mA, not less than about 12.1 mA, not less than about 12.2 mA, not less than about 12.3 mA, not less than about 12.4 mA, not less than about 12.5 mA, not less than about 12.6 mA, not less than about 12.7 mA, not less than about 12.8 mA, not less than about 12.9 mA, not less than about 13.0 mA, not less than about 13.1 mA, not less than about 13.2 mA, not less than about 13.3 mA, not less than about 13.4 mA, not less than about 13.5 mA, not less than about 13.6 mA, not less than about 13.7 mA, not less than about 13.8 mA, not less than about 13.9 mA, not less than about 14.0 mA, not less than about 14.1 mA, not less than about 14.2 mA, not less than about 14.3 mA, not less than about 14.4 mA, not less than about 14.5 mA, not less than about 14.6 mA, not less than about 14.7 mA, not less than about 14.8 mA, not less than about 14.9 mA, not less than about 15.0 mA, not less than about 15.1 mA, not less than about 15.2 mA, not less than about 15.3 mA, not less than about 15.4 mA, not less than about 15.5 mA, not less than about 15.6 mA, not less than about 15.7 mA, not less than about 15.8 mA, and the like.

In some embodiments the electrodes or microcells can comprise a clear conductive material. For example, in certain embodiments indium tin oxide (ITO) can be used. In other embodiments other transparent conductive oxides (TCOs), conductive polymers, metal grids, carbon nanotubes, graphene, and nanowire thin films can be employed.

In certain embodiments, reservoir or electrode geometry can comprise shapes including circles, polygons, lines, zigzags, ovals, stars, or any suitable variety. This provides the ability to design/customize surface electric field shapes as well as depth of penetration. For example, in embodiments it can be desirable to employ an electric field of greater strength or depth to achieve optimal treatment.

Reservoir or electrode or dot sizes and concentrations can vary, as these variations can allow for changes in the properties of the electric field created by embodiments of the invention. Certain embodiments provide an electric field at about, for example, 0.5-5.0 V at the device surface under normal tissue loads with resistance of 100 to 100K ohms.

In embodiments, disclosed devices can provide an electric field of greater than physiological strength, for example to a depth of, at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or more.

In various embodiments dissimilar metals can be used to create a customized electric field with a desired voltage or microcurrent. In certain embodiments the pattern of reservoirs can control the watt density and shape of the electric field. For example. In embodiments it can be desirable to employ an electric field of greater strength or depth in an area where skin is thicker to achieve optimal treatment.

In embodiments devices disclosed herein the electric field or current or both applied to a tissue can be designed or produced or adjusted based upon feedback from the tissue or upon an algorithm within sensors operably connected to the embodiment and a control module. The electric field, electric current, or both can be stronger in one zone and weaker in another. The electric field, electric current, or both can change with time and be modulated based on treatment goals or feedback from the tissue or patient. The control module can monitor and adjust the size, strength, density, shape, or duration of electric field or electric current based on tissue parameters. For example, embodiments disclosed herein can produce and maintain localized electrical events. For example, embodiments disclosed herein can produce specific values for the electric field duration, electric field size, electric field shape, field depth, current, polarity, and/or voltage of the device or system.

As described, the disclosed systems and devices can include a backing sheet or base layer. The backing sheet or base layer can be useful in reducing the amount of motion between tissue and device and/or can be a substrate for the multi-array matrix of biocompatible microcells and the hydrogel(s). In some embodiments, this backing sheet can be elastic. In other embodiments, the backing sheet can include components such as straps to maintain or help maintain its position. In some embodiments, the backing sheet can comprise a strap on either end of the long axis, or a strap linking on end of the long axis to the other. The straps can comprise velcro, snaps, or a similar fastening system. In further embodiments the strap can comprise a conductive material, for example a wire to electrically link the device with other components, such as monitoring equipment or a power source.

In further embodiments the strap can comprise a conductive material, for example a wire to electrically link the device with other components, such as monitoring equipment or a power source. In embodiments the device can be wirelessly linked to monitoring or data collection equipment, for example linked via Bluetooth to a cell phone or computer that collects data from the device. In certain embodiments the device can comprise data collection means, such as temperature, pH, pressure, or conductivity data collection means. In certain embodiments, disclosed devices and systems can comprise data collection means, such as temperature, pH, pressure, or conductivity data collection means. Embodiments can comprise a display, for example to visually present, for example, the temperature, pH, pressure, or conductivity data to a user. Embodiments can include, for example, tracking equipment so as to track and/or quantify a user's movements or performance. Embodiments can include, for example, an accelerometer, so as to measure impact forces on a user.

In some embodiments, the backing sheet or base layer can also include a component such as an adhesive to maintain or help maintain its position. The adhesive component can be covered with a protective layer that is removed to expose the adhesive at the time of use. In embodiments the adhesive can comprise, for example, sealants, such as hypoallergenic sealants, waterproof sealants such as epoxies, and the like.

If elastic is used in the backing sheet, it can include an elastic film with elasticity, for example, similar to that of skin, or greater than that of skin, or less than that of skin. In embodiments, the LLEC or LLEF system can comprise a laminate where layers of the laminate can be of varying elasticities. For example, an outer layer may be highly elastic and an inner layer in-elastic or less elastic. The in-elastic layer can be made to stretch by placing stress relieving discontinuous regions or slits through the thickness of the material so there is a mechanical displacement rather than stress that would break the fabric weave before stretching would occur. In embodiments the slits can extend completely through a layer or the system or can be placed where expansion is required. In embodiments of the system the slits do not extend all the way through the system or a portion of the system such as the substrate. In embodiments the discontinuous regions can pass halfway through the long axis of the substrate.

In embodiments the backing sheet can be shaped to fit an area of desired use or treatment. In some embodiments, a device can be supplied as a large sheet and cut to a particular shape for use.

In some embodiments, the backing sheet or base layer can be a bandage. If provided as a bandage, the bandage can include any or all of the features described herein.

In some embodiments, the backing sheet can be a substrate such as a fabric, a fiber, or the like. In embodiments the substrate can be pliable, for example to better follow the contours of an area to be treated, such as the face or back. In embodiments the substrate can comprise a gauze or mesh or plastic. Suitable types of pliable substrates for use in embodiments disclosed herein can be absorbent or non-absorbent textiles, low-adhesives, vapor permeable films, hydrocolloids, alginates, foams, foam-based materials, cellulose-based materials including Kettenbach fibers, hollow tubes, fibrous materials, such as those impregnated with anhydrous/hygroscopic materials, beads and the like, or any suitable material as known in the art.

In some embodiments, the backing sheet or base layer can comprise “anchor” regions or “arms” or straps to affix the system securely. For example, a system or device as described herein can be secured to or around a curved surface, and anchor regions of the backing sheet can extend to areas of minimal stress or movement to securely affix the system in place.

A hydrogel can be a network of polymer chains that are hydrophilic. Hydrogels can be highly absorbent natural or synthetic polymeric networks. Hydrogels can be configured to contain a high percentage of water (for example, they can contain over 90% water) in a hydrated state or can be de-hydrated to remove the water content from the hydrogel.

Hydrogels can possess a degree of flexibility very similar to natural tissue. In some embodiments, this flexibility can be due to their water content. In some embodiments, this flexibility can provide a coated multi-array matrix of biocompatible microcells that retains substantially all the flexibility of a multi-array matrix of biocompatible microcells without a hydrogel. Substantially all of the flexibility includes devices that retain more than about 80%, more than about 85%, more than about 90%, more than about 95%, or more than about 99% of the flexibility of the device without a hydrogel. In some embodiments, a device with a hydrogel retains all the flexibility of a device without a hydrogel.

As described, the devices described herein can comprise at least one hydrogel that coats or otherwise impregnates the multi-array matrix of biocompatible microcells of the device. A hydrogel as described herein can include any hydrogel known in the art that can provide rehydration characteristics that allow bioelectric devices as described herein to function as if the hydrogel were not present or substantially as if the hydrogel were not present, yet keep the microcell batteries activated for an extended time as if an amorphous hydrogel were applied at time of use. In embodiments, the hydrogel can coat the matrix present on the base layer. In further embodiments, the hydrogel can comprise the matrix.

Further, the hydrogel can function to retain or “lock” the eventual rehydration voltages and/or amperages that provide localized treatment.

Suitable hydrogels can include, but are not limited to polyvinyl alcohol, sodium polyacrylate, acrylate based polymers, glycolated polymers, cellulose, glycerol, sugars, agarose, methylcellulose, hyaluronan, other naturally derived polymers, and combinations thereof.

A hydrogel can be configured in a variety of viscosities. Viscosity is a measurement of a fluid or material's resistance to gradual deformation by shear stress or tensile stress. In embodiments the electrical field can be extended through a semi-liquid hydrogel with a low viscosity. In other embodiments the electrical field can be extended through a solid hydrogel with a high viscosity.

In some embodiments, the hydrogel(s) described herein may be configured to have a viscosity of between about 0.5 Pa·s and greater than about 10¹² Pa·s. In embodiments, the viscosity of a hydrogel can be, for example, between 0.5 and 10¹² Pa·s, between 1 Pa·s and 10⁶ Pa·s, between 5 and 10³ Pa·s, between 10 and 100 Pa·s, between 15 and 90 Pa·s, between 20 and 80 Pa·s, between 25 and 70 Pa·s, between 30 and 60 Pa·s, or the like when applied to a device.

The hydrogel can be supplied in a device as described herein as an amount of hydrogel per square foot of system or device. In some embodiments, about 1 g, about 5 g, about 10 g, about 15 g, about 20 g, about 25 g, about 30 g, about 35 g, about 40 g, about 45 g, about 50 g, at least about 1 g, at least about 5 g, at least about 10 g, at least about 20 g, between about 1 g and about 20 g, between about 10 g and about 20 g or between about 15 g and about 25 g of a hydrogel per square foot of device can be sufficient to provide the herein desired results.

The hydrogels or coatings of the hydrogels can include active agents, for example hypoallergenic agents, drugs, biologics, stem cells, growth factors, skin substitutes, cosmetic products, combinations, or combinations thereof, or the like. Stem cells can include, for example, embryonic stem cells, bone-marrow stem cells, adipose stem cells, and the like.

A growth factor is a naturally-occurring substance capable of stimulating cellular growth, proliferation, healing, and cellular differentiation, often a protein or a steroid hormone. Growth factors are important for regulating a variety of cellular processes. Growth factors typically act as signaling molecules between cells. Examples are cytokines and hormones that bind to specific receptors on the surface of their target cells. They often promote cell differentiation and maturation, which varies between growth factors. For example, bone morphogenetic proteins stimulate bone cell differentiation, while fibroblast growth factors and vascular endothelial growth factors stimulate blood vessel differentiation.

Growth factors can include, for example, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor 1 or 2 (FGF-1 or -2), Fetal Bovine Somatotrophin (FBS), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Keratinocyte growth factor (KGF), Migration-stimulating factor (MSF), Myostatin (GDF-8), Nerve growth factor (NGF) and other neurotrophins, Platelet-derived growth factor (PDGF), Thrombopoietin (TPO), T-cell growth factor (TCGF), Transforming growth factor alpha (TGF-α), Transforming growth factor beta (TGF-β), Tumor necrosis factor-alpha (TNF-α), Vascular endothelial growth factor (VEGF), Wnt Signaling Pathway, Placental growth factor (PGF), IL-1, IL-2, IL-3, IL-4, IL-5, IL-6, IL-7, Renalase, or combinations thereof. Active agents can include alpha granules.

Cosmetic products can include, for example, moisturizers, exfoliants, antioxidants, and the like.

Drugs can include but are not limited to anti-inflammatories, painkillers, antibiotics, antivirals, and wound treatment compositions. These active agents can be mixed with the hydrogel prior to application to a multi-array matrix of biocompatible microcells or can be otherwise attached to the hydrogel when the hydrogel is already part of a device, such as by chemical substitution or through the use of intermolecular forces. In embodiments the active agent can be applied to an area of treatment prior to contacting the area with a system or device disclosed herein.

In embodiments, devices and systems disclosed herein can produce a low level micro-current to a treatment site once hydrated from the dehydrated state. Generally, devices are provided in a dehydrated state and then subsequently hydrated before application by a user or practitioner.

Dissimilar conductive metals used to make a LLEC or LLEF system disclosed herein can be silver and zinc, and the electrolytic solution can include sodium chloride in water. In certain embodiments the electrodes are applied onto a non-conductive surface to create a pattern, most preferably an array or multi-array of voltaic cells that do not spontaneously react until they contact an electrolytic solution. Sections of this description use the terms “printing” with “ink,” but it is to be understood that the patterns may also be “painted” with “paints.” The use of any suitable means for applying a conductive material is contemplated. In embodiments, “ink” or “paint” can comprise any material such as a solution suitable for forming an electrode on a surface such as a conductive material including a conductive metal solution. In embodiments “printing” or “painted” can comprise any method of applying a solution to a material upon which a matrix is desired, for example a transparent or translucent material.

The applied electrodes or reservoirs or dots can adhere or bond to the primary surface or substrate because a biocompatible binder is mixed, in embodiments into separate mixtures, with each of the dissimilar metals that will create the pattern of voltaic cells, in embodiments. Most inks are simply a carrier, and a binder mixed with pigment. Similarly, conductive metal solutions can be a binder mixed with a conductive element. The resulting conductive metal solutions can be used with an application method such as screen printing to apply the electrodes to the primary surface in predetermined patterns. Once the conductive metal solutions dry and/or cure, the patterns of spaced electrodes can substantially maintain their relative position, even on a flexible material such as that used for a LLEC or LLEF system. The conductive metal solution can be allowed to dry before being applied to a surface so that the conductive materials do not mix, which could interrupt the array and cause direct reactions that will release the elements.

In certain embodiments that utilize a poly-cellulose binder, the binder itself can have a beneficial effect such as reducing the local concentration of matrix metallo-proteases through an iontophoretic process that drives the cellulose into the surrounding tissue. This process can be used to electronically drive other components such as drugs into the surrounding tissue.

The binder can comprise any biocompatible liquid material that can be mixed with a conductive element (preferably metallic crystals of silver or zinc) to create a conductive solution which can be applied as a thin coating to a microsphere. One suitable binder is a solvent reducible polymer, such as the polyacrylic non-toxic silk-screen ink manufactured by COLORCON® Inc., a division of Berwind Pharmaceutical Services, Inc. (see COLORCON® NO-TOX® product line, part number NT28). In an embodiment the binder is mixed with high purity (at least 99.99%, in an embodiment) metallic silver crystals to make the silver conductive solution. The silver crystals, which can be made by grinding silver into a powder, are preferably smaller than 100 microns in size or about as fine as flour. In an embodiment, the size of the crystals is about 325 mesh, which is typically about 40 microns in size or a little smaller. The binder is separately mixed with high purity (at least 99.99%, in an embodiment) metallic zinc powder which has also preferably been sifted through standard 325 mesh screen, to make the zinc conductive solution.

Other powders of metal can be used to make other conductive metal solutions in the same way as described in other embodiments.

The size of the metal crystals, the availability of the surface to the conductive fluid and the ratio of metal to binder affects the release rate of the metal from the mixture. When COLORCON® polyacrylic ink is used as the binder, about 10 to 40 percent of the mixture should be metal for a long term bandage (for example, one that stays on for about 10 days). If the same binder is used, but the percentage of the mixture that is metal is increased to 60 percent or higher, a typical system will be effective for longer. For example, for a longer term device, the percent of the mixture that should be metal can be 40 percent, or 42 percent, 44 percent, 46 percent, 48 percent, 50 percent, 52 percent, 54 percent, 56 percent, 58 percent, 60 percent, 62 percent, 64 percent, 66 percent, 68 percent, 70 percent, 72 percent, 74 percent, 76 percent, 78 percent, 80 percent, 82 percent, 84 percent, 86 percent, 88 percent, 90 percent, or the like.

For LLEC or LLEF systems comprising a pliable substrate it can be desired to decrease the percentage of metal down to, for example, 20 percent, 18 percent, 16 percent, 14 percent, 12 percent, 10 percent, 5 percent, or less, or to use a binder that causes the crystals to be more deeply embedded, so that the primary surface will be antimicrobial for a very long period of time and will not wear prematurely. Other binders can dissolve or otherwise break down faster or slower than a polyacrylic ink, so adjustments can be made to achieve the desired rate of spontaneous reactions from the voltaic cells.

To maximize the number of voltaic cells, in various embodiments, a pattern of alternating silver masses or electrodes or reservoirs and zinc masses or electrodes or reservoirs can create an array of electrical currents across the primary surface or base layer. A basic pattern, shown in FIG. 1, has each mass of silver equally spaced from four masses of zinc, and has each mass of zinc equally spaced from four masses of silver, according to an embodiment. The first electrode 6 is separated from the second electrode 10 by a spacing 8. The designs of first electrode 6 and second electrode 10 are simply round dots, and in an embodiment, are repeated. Numerous repetitions 12 of the designs result in a pattern. For an exemplary device comprising silver and zinc, each silver design preferably has about twice as much mass as each zinc design, in an embodiment. For the pattern in FIG. 1, the silver designs are most preferably about a millimeter from each of the closest four zinc designs, and vice-versa. The resulting pattern of dissimilar metal masses defines an array of voltaic cells when introduced to an electrolytic solution. Further disclosure relating to methods of producing micro-arrays can be found in U.S. Pat. No. 7,813,806 entitled CURRENT PRODUCING SURFACE FOR TREATING BIOLOGIC TISSUE issued Oct. 12, 2010, which is incorporated by reference in its entirety.

A dot pattern of masses like the alternating round dots of FIG. 1 can be preferred when applying conductive material onto a flexible base layer, such as those used for an article of clothing such as a shirt, shorts, sleeves, or socks, as the dots won't significantly affect the flexibility of the material. To maximize the density of electrical current over a primary surface the pattern of FIG. 2 can be used. The first electrode 6 in FIG. 2 is a large hexagonally shaped dot, and the second electrode 10 is a pair of smaller hexagonally shaped dots that are spaced from each other. The spacing 8 that is between the first electrode 6 and the second electrode 10 maintains a relatively consistent distance between adjacent sides of the designs. Numerous repetitions 12 of the designs result in a pattern 14 that can be described as at least one of the first design being surrounded by six hexagonally shaped dots of the second design.

FIGS. 3 and 4 show how the pattern of FIG. 2 can be used to make an embodiment disclosed herein. The pattern shown in detail in FIG. 2 is applied to the primary surface 2 of an embodiment. The back 20 of the printed material is fixed to a substrate layer 22. This layer is adhesively fixed to a pliable layer 16.

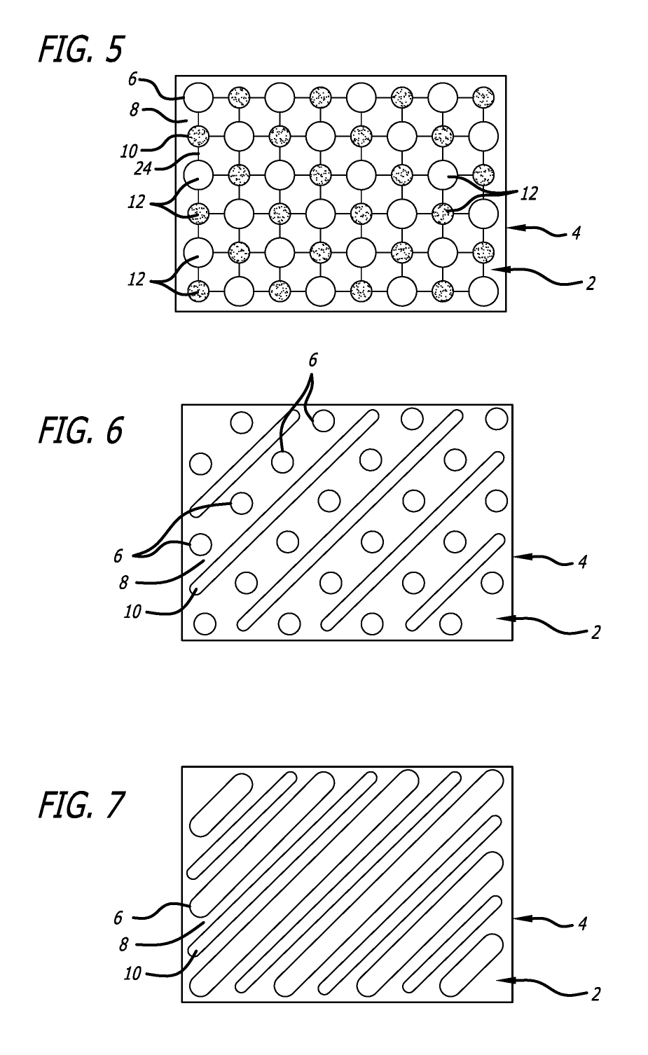

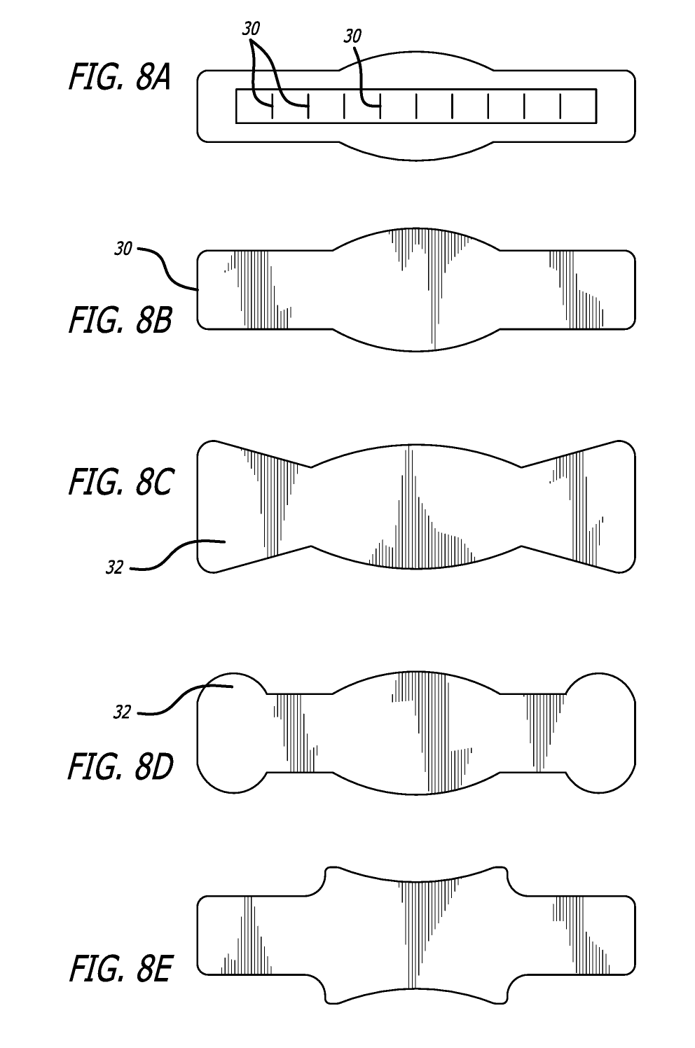

FIG. 5 shows an additional feature, which can be added between designs, that can initiate the flow of current in a poor electrolytic environment. A fine line 24 is printed using one of the conductive metal solutions along a current path of each voltaic cell. The fine line will initially have a direct reaction but will be depleted until the distance between the electrodes increases to where maximum voltage is realized. The initial current produced is intended to help control edema so that the LLEC system will be effective. If the electrolytic solution is highly conductive when the system is initially applied the fine line can be quickly depleted and the device will function as though the fine line had never existed.

FIGS. 6 and 7 show alternative patterns that use at least one line design. The first electrode 6 of FIG. 6 is a round dot similar to the first design used in FIG. 1. The second electrode 10 of FIG. 6 is a line. When the designs are repeated, they define a pattern of parallel lines that are separated by numerous spaced dots. FIG. 7 uses only line designs. The first electrode 6 can be thicker or wider than the second electrode 10 if the oxidation-reduction reaction requires more metal from the first conductive element (mixed into the first design's conductive metal solution) than the second conductive element (mixed into the second design's conductive metal solution). The lines can be dashed. Another pattern can be silver grid lines that have zinc masses in the center of each of the cells of the grid. The pattern can be letters printed from alternating conductive materials so that a message can be printed onto the primary surface-perhaps a brand name or identifying information such as patient blood type.

Because the spontaneous oxidation-reduction reaction of an embodiment utilizing silver and zinc uses a ratio of approximately two silver atoms to one zinc atom, the silver design can contain about twice as much mass as the zinc design in an embodiment. At a spacing of about 1 mm between the closest dissimilar metals (closest edge to closest edge) each voltaic cell that contacts a conductive fluid such as a cosmetic cream can create approximately 1 volt of potential that will penetrate substantially through its surrounding surfaces. Closer spacing of the dots can decrease the resistance, providing less potential, and the current will not penetrate as deeply. Therefore, spacing between the closest conductive materials on the base layer or substrate can be, for example, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, or the like.

In certain embodiments the spacing between the closest conductive materials on the base layer can be not more than 0.1 mm, not more than 0.2 mm, not more than 0.3 mm, not more than 0.4 mm, not more than 0.5 mm, not more than 0.6 mm, not more than 0.7 mm, not more than 0.8 mm, not more than 0.9 mm, not more than 1 mm, not more than 1.1 mm, not more than 1.2 mm, not more than 1.3 mm, not more than 1.4 mm, not more than 1.5 mm, not more than 1.6 mm, not more than 1.7 mm, not more than 1.8 mm, not more than 1.9 mm, not more than 2 mm, not more than 2.1 mm, not more than 2.2 mm, not more than 2.3 mm, not more than 2.4 mm, not more than 2.5 mm, not more than 2.6 mm, not more than 2.7 mm, not more than 2.8 mm, not more than 2.9 mm, not more than 3 mm, not more than 3.1 mm, not more than 3.2 mm, not more than 3.3 mm, not more than 3.4 mm, not more than 3.5 mm, not more than 3.6 mm, not more than 3.7 mm, not more than 3.8 mm, not more than 3.9 mm, not more than 4 mm, not more than 4.1 mm, not more than 4.2 mm, not more than 4.3 mm, not more than 4.4 mm, not more than 4.5 mm, not more than 4.6 mm, not more than 4.7 mm, not more than 4.8 mm, not more than 4.9 mm, not more than 5 mm, not more than 5.1 mm, not more than 5.2 mm, not more than 5.3 mm, not more than 5.4 mm, not more than 5.5 mm, not more than 5.6 mm, not more than 5.7 mm, not more than 5.8 mm, not more than 5.9 mm, not more than 6 mm, or the like.

In certain embodiments spacing between the closest conductive materials on the base layer can be not less than 0.1 mm, or not less than 0.2 mm, not less than 0.3 mm, not less than 0.4 mm, not less than 0.5 mm, not less than 0.6 mm, not less than 0.7 mm, not less than 0.8 mm, not less than 0.9 mm, not less than 1 mm, not less than 1.1 mm, not less than 1.2 mm, not less than 1.3 mm, not less than 1.4 mm, not less than 1.5 mm, not less than 1.6 mm, not less than 1.7 mm, not less than 1.8 mm, not less than 1.9 mm, not less than 2 mm, not less than 2.1 mm, not less than 2.2 mm, not less than 2.3 mm, not less than 2.4 mm, not less than 2.5 mm, not less than 2.6 mm, not less than 2.7 mm, not less than 2.8 mm, not less than 2.9 mm, not less than 3 mm, not less than 3.1 mm, not less than 3.2 mm, not less than 3.3 mm, not less than 3.4 mm, not less than 3.5 mm, not less than 3.6 mm, not less than 3.7 mm, not less than 3.8 mm, not less than 3.9 mm, not less than 4 mm, not less than 4.1 mm, not less than 4.2 mm, not less than 4.3 mm, not less than 4.4 mm, not less than 4.5 mm, not less than 4.6 mm, not less than 4.7 mm, not less than 4.8 mm, not less than 4.9 mm, not less than 5 mm, not less than 5.1 mm, not less than 5.2 mm, not less than 5.3 mm, not less than 5.4 mm, not less than 5.5 mm, not less than 5.6 mm, not less than 5.7 mm, not less than 5.8 mm, not less than 5.9 mm, not less than 6 mm, or the like.

Disclosures of the present specification include LLEC or LLEF systems comprising a primary surface of a material wherein the material is adapted to be applied to an area of tissue such as a muscle; a first electrode design formed from a first conductive liquid that includes a mixture of a polymer and a first element, the first conductive liquid being applied into a position of contact with the primary surface, the first element including a metal species, and the first electrode design including at least one dot or reservoir, wherein at least one of the at least one dot or reservoir has approximately a 1.5 mm+1-1 mm mean diameter; a second electrode design formed from a second conductive liquid that includes a mixture of a polymer and a second element, the second element including a different metal species than the first element, the second conductive liquid being printed into a position of contact with the primary surface, and the second electrode design including at least one other dot or reservoir, wherein at least one of the at least one other dot or reservoir has approximately a 2.5 mm+1-2 mm mean diameter; a spacing on the primary surface that is between the first electrode design and the second electrode design such that the first electrode design does not physically contact the second electrode design, wherein the spacing is approximately 1.5 mm+1-1 mm, and at least one repetition of the first electrode design and the second electrode design, the at least one repetition of the first electrode design being substantially adjacent the second electrode design, wherein the at least one repetition of the first electrode design and the second electrode design, in conjunction with the spacing between the first electrode design and the second electrode design, defines at least one pattern of at least one voltaic cell for spontaneously generating at least one electrical current when introduced to an electrolytic solution. Therefore, electrodes, dots or reservoirs can have a mean diameter of 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, or the like.

In further embodiments, electrodes, dots or reservoirs can have a mean diameter of not less than 0.2 mm, not less than 0.3 mm, not less than 0.4 mm, not less than 0.5 mm, not less than 0.6 mm, not less than 0.7 mm, not less than 0.8 mm, not less than 0.9 mm, not less than 1.0 mm, not less than 1.1 mm, not less than 1.2 mm, not less than 1.3 mm, not less than 1.4 mm, not less than 1.5 mm, not less than 1.6 mm, not less than 1.7 mm, not less than 1.8 mm, not less than 1.9 mm, not less than 2.0 mm, not less than 2.1 mm, not less than 2.2 mm, not less than 2.3 mm, not less than 2.4 mm, not less than 2.5 mm, not less than 2.6 mm, not less than 2.7 mm, not less than 2.8 mm, not less than 2.9 mm, not less than 3.0 mm, not less than 3.1 mm, not less than 3.2 mm, not less than 3.3 mm, not less than 3.4 mm, not less than 3.5 mm, not less than 3.6 mm, not less than 3.7 mm, not less than 3.8 mm, not less than 3.9 mm, not less than 4.0 mm, not less than 4.1 mm, not less than 4.2 mm, not less than 4.3 mm, not less than 4.4 mm, not less than 4.5 mm, not less than 4.6 mm, not less than 4.7 mm, not less than 4.8 mm, not less than 4.9 mm, not less than 5.0 mm, or the like.

In further embodiments, electrodes, dots or reservoirs can have a mean diameter of not more than 0.2 mm, not more than 0.3 mm, not more than 0.4 mm, not more than 0.5 mm, not more than 0.6 mm, not more than 0.7 mm, not more than 0.8 mm, not more than 0.9 mm, not more than 1.0 mm, not more than 1.1 mm, not more than 1.2 mm, not more than 1.3 mm, not more than 1.4 mm, not more than 1.5 mm, not more than 1.6 mm, not more than 1.7 mm, not more than 1.8 mm, not more than 1.9 mm, not more than 2.0 mm, not more than 2.1 mm, not more than 2.2 mm, not more than 2.3 mm, not more than 2.4 mm, not more than 2.5 mm, not more than 2.6 mm, not more than 2.7 mm, not more than 2.8 mm, not more than 2.9 mm, not more than 3.0 mm, not more than 3.1 mm, not more than 3.2 mm, not more than 3.3 mm, not more than 3.4 mm, not more than 3.5 mm, not more than 3.6 mm, not more than 3.7 mm, not more than 3.8 mm, not more than 3.9 mm, not more than 4.0 mm, not more than 4.1 mm, not more than 4.2 mm, not more than 4.3 mm, not more than 4.4 mm, not more than 4.5 mm, not more than 4.6 mm, not more than 4.7 mm, not more than 4.8 mm, not more than 4.9 mm, not more than 5.0 mm, or the like.

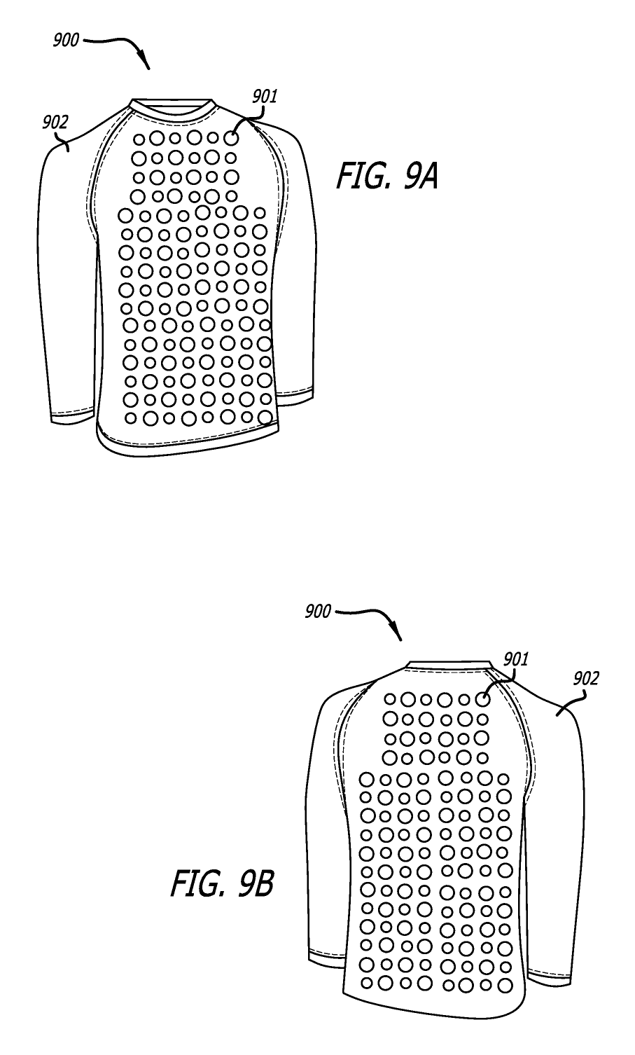

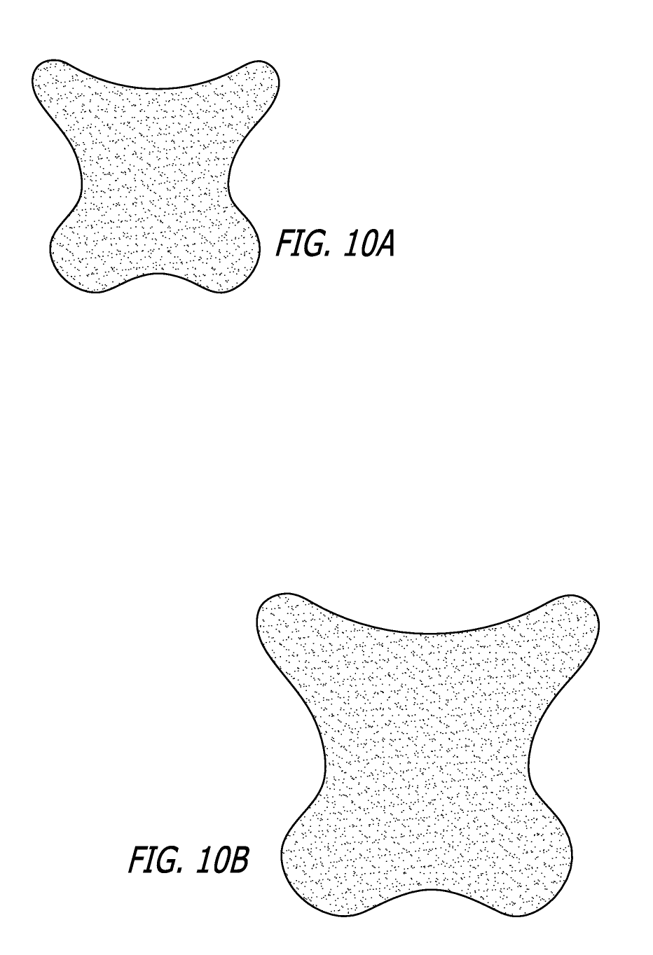

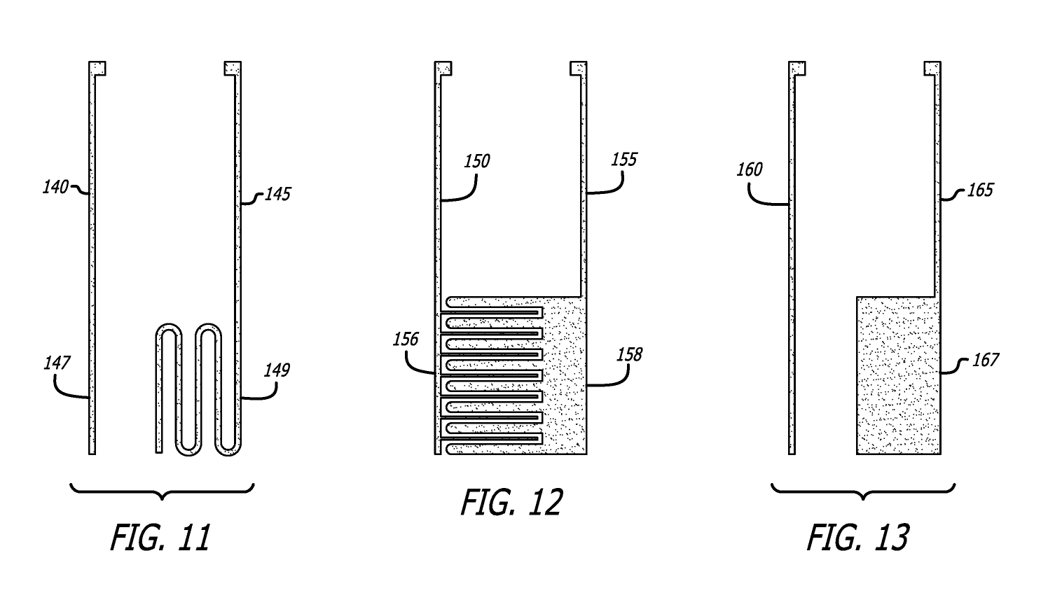

FIG. 9A depicts an example garment 900 comprising a multi-array matrix of biocompatible microcells. Garment 900 comprises electrodes 901 and substrate 902. Electrodes 901 are printed around the entirety of substrate 902 including the back of garment 910. Electrodes 901 can provide a LLEF to tissue, and, when in contact with a conductive material, a LLEC. In another embodiment, electrodes 901 can be printed to a portion of the garment 900, as depicted in FIG. 9B. For example, electrodes 901 can be applied to only the back of garment 900 to provide a LLEF to lower back. In certain embodiment, electrodes 901 can also be removed and a new set of dots 901 can be applied to similar or new location on garment (900). The array can be printed or applied such that it contacts the skin while in use. For example, the array can be printed on or applied to the inside of the garment.

FIG. 11 shows an embodiment utilizing two electrodes (one positive and one negative). Upper arms 140 and 145 can be, for example, 1, 2, 3, or 4 mm in width. Lower arm 147 and serpentine 149 can be, for example, 1, 2, 3, or 4 mm in width. The electrodes can be, for example, 1, 2, or 3 mm in depth.

FIG. 12 shows an embodiment utilizing two electrodes (one positive and one negative). Upper arms 150 and 155 can be, for example, 1, 2, 3, or 4 mm in width. The extensions protruding from the lower arm 156 can be, for example, 1, 1.5, 2, 2.5, 3, 3.5, or 4 mm in width. The extensions protruding from the comb 158 can be, for example, 1, 2, 3, 4, 5, 6, or 7 mm in width. The electrodes can be, for example, 1, 2, or 3 mm in depth.

FIG. 13 shows an embodiment utilizing two electrodes (one positive and one negative). Upper arms 160 and 165 can be, for example, 1, 2, 3, or 4 mm in width. Lower block 167 can be, for example, 30, 32, 34, 36, 38, 40, 42, 44, 46, 48, 50, 52, or 54 mm along its shorter axis. Lower block 167 can be, for example, 60, 65, 70, 75, 80, 85, 90, 95, or 100 mm along its longer axis. The electrodes can be, for example, 1, 2, or 3 mm in depth.

In embodiments such as those in FIGS. 11-13, the width and depth of the various areas of the electrode can be designed to produce a particular electric field, or, when both electrodes are in contact with a conductive material, a particular electric current. For example, the width of the various areas of the electrode can be, for example, 0.1 mm, or 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, or 7 mm, or 8 mm, or 9 mm, or 10 mm, or 11 mm, or the like.

In embodiments, the depth or thickness of the various areas of the electrode can be, for example, 0.1 mm, or 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, or the like.

The shortest distance between the two electrodes in an embodiment can be, for example, 0.1 mm, or 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, or the like.

In embodiments, the length of the long axis of the device can be, for example, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 75 mm, 100 mm, 150 mm, 200 mm, 250 mm, 300 mm, 350 mm, 400 mm, 450 mm, 500 mm, 600 mm, 700 mm, 800 mm, 900 mm, 1000 mm, or more, or the like.

In embodiments, the length of the short axis of the device can be, for example, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 75 mm, 100 mm, or more, or the like.

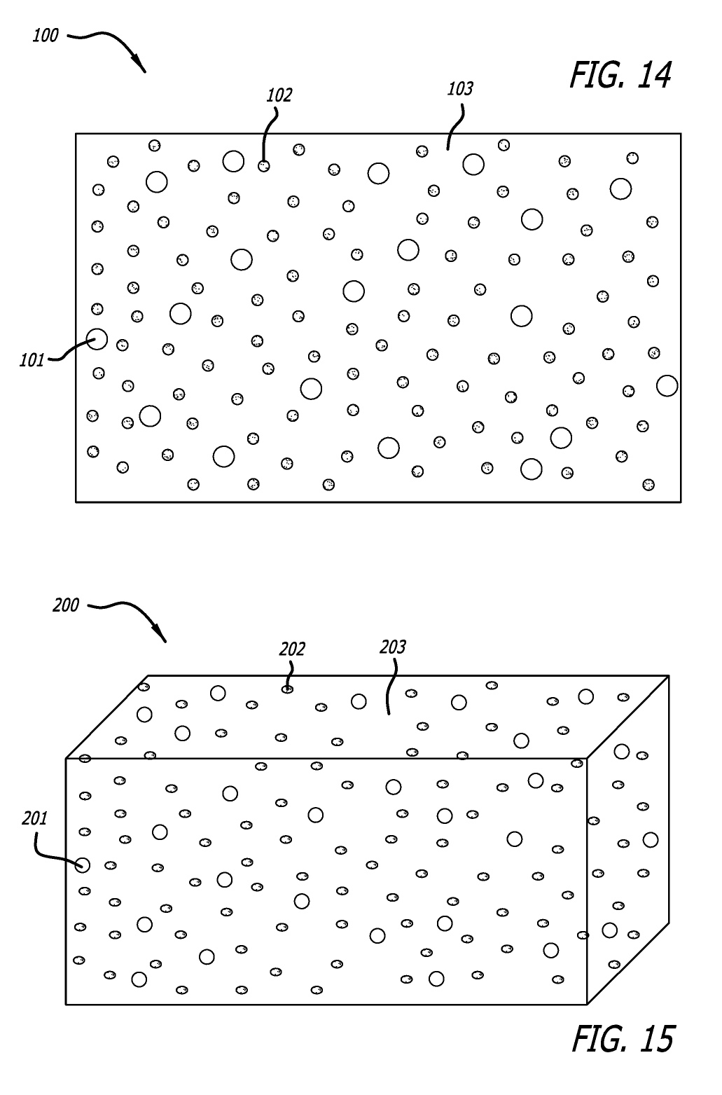

FIG. 14 depicts a graphical representation of a bioelectric hydrogel comprising a matrix according to one or more embodiments. In FIG. 14, the dissimilar first electrode 101 and second electrode 102 are in a desired hydrophilic polymer base 103 of a hydrogel 100, for example an ointment or cellular culture medium. In one embodiment a hydrogel 100 is a material of a LLEC or LLEF system that comes into direct contact with an area to be treated such as a skin surface or within the hydrogel for cellular culture. Hydrogel 100 can also be configured or shaped into a three dimensional object or material as shown in FIG. 15. In FIG. 15, the dissimilar first electrode 201 and second electrode 202 are coupled into a desired hydrophilic polymer base 203 of a hydrogel 200. First electrode 201 and second electrode 202 can be placed within hydrophilic polymer base 203 as needed to accommodate the desired use.

To maximize the number of voltaic cells, in various embodiments, a “pattern” (in some hydrogels, the positions of the electrodes can change) of alternating silver masses (e.g., 101 as shown in FIG. 14) or electrodes or reservoirs and zinc masses (e.g., 102 as shown in FIG. 14) or electrodes or reservoirs can create an array of electrical currents across the hydrogel. A basic embodiment, shown in FIG. 14, has each mass of silver randomly spaced from masses of zinc, and has each mass of zinc randomly spaced from masses of silver, according to an embodiment. In another embodiment, mass of silver can be equally spaced from masses of zinc, and has each mass of zinc equally spaced from masses of silver. That is, the electrodes or reservoirs or dots can either be a uniform pattern, a random pattern, or a combination of the like. The first electrode 101 is separated from the second electrode 102 by a hydrophilic polymer base 103. The designs of first electrode 101 and second electrode 102 are simply round dots, and in an embodiment, are repeated throughout the hydrogel. For an exemplary device comprising silver and zinc, each silver design preferably has about twice as much mass as each zinc design, in an embodiment. For the embodiment in FIG. 14, the silver designs are most preferably about a millimeter from each of the closest four zinc designs, and vice-versa. The resulting pattern of dissimilar metal masses defines an array of voltaic cells when introduced to an electrolytic solution.

The material concentrations or quantities within and/or the relative sizes (e.g., dimensions or surface area) of the first and second reservoirs can be selected deliberately to achieve various characteristics of the systems' behavior. For example, the quantities of material within a first and second reservoir can be selected to provide an apparatus having an operational behavior that depletes at approximately a desired rate and/or that “dies” after an approximate period of time after activation. In an embodiment the one or more first reservoirs and the one or more second reservoirs are configured to sustain one or more currents for an approximate pre-determined period of time, after activation. It is to be understood that the amount of time that currents are sustained can depend on external conditions and factors (e.g., the quantity and type of activation material), and currents can occur intermittently depending on the presence or absence of activation material. Further disclosure relating to producing reservoirs that are configured to sustain one or more currents for an approximate pre-determined period of time can be found in U.S. Pat. No. 7,904,147 entitled SUBSTANTIALLY PLANAR ARTICLE AND METHODS OF MANUFACTURE issued Mar. 8, 2011, which is incorporated by reference herein in its entirety.

In embodiments that include very small reservoirs (e.g., on the nanometer scale), the difference of the standard potentials can be substantially less or more.

Methods of applying the herein described hydrogels to a bioelectric device are also described. In one embodiment, the described hydrogels can be applied to a multi-array matrix of biocompatible microcells included on a base sheet (bioelectric device sheet) using a number of different techniques. Application techniques can include but are not limited to spray coating, dipping, brushing, rolling, sprinkling, vapor depositing, and the like or a combination thereof.

In one embodiment, a coating of a hydrogel can be manually spread on the active side of a bioelectric device sheet such as on a multi-array matrix of biocompatible microcells. This process can be accomplished using a coating system similar to one used in silkscreening.

In some embodiments, the hydrogel can be thinned by adding additional water to the hydrogel before application. When the viscosity of the hydrogel has been reduced sufficiently, the thinned hydrogel can be applied. A reduced viscosity hydrogel can be used for dip coating.

In another embodiment, a hydrogel can be sprayed onto a multi-array matrix of biocompatible microcells in a manner similar to spray painting. In some embodiments, a thinned hydrogel can be used for spraying.

If a hydrogel is applied using a sprinkling technique, dry components of a hydrogel can be sprinkled on the surface of a multi-array matrix of biocompatible microcells, and the dry component/sheet combination can be lightly sprayed with water to hold the particles in place. The moist components can then be dried.

After application of the hydrogel, the sheets can then be dried using any conventional drying technique. Drying can be accomplished by air drying, hang drying, drying in an oven, drying under a powered dryer, vacuum drying, or the like. In one embodiment, coated sheets can be hung up to dry. In one embodiment, a coated sheet can be placed on a surface and allowed to air dry. In other embodiments, drying times may be decreased by placing the coated sheets in an oven. Oven drying of coated sheets can be performed in batch mode or in a continuous mode. Further, drying times can further be decreased using a vacuum oven for drying.

The dried sheets can be rehydrated for use. When rehydrated, the sheets can attain their intended properties included voltages. Rehydration can be actively accomplished using water saline, or other appropriate liquid. Actively rehydrating can be accomplished by spraying, dipping, brushing, rolling, and the like or a combination thereof.

In some embodiments, the dehydrated sheet can be dipped in an appropriate liquid. Dipping can require a dwell time in the liquid to properly hydrate the sheet. Dwell times can be about 1 second, about 2 seconds, about 3 seconds, about 4 seconds, about 5 seconds, about 10 seconds, about 20 seconds, about 30 seconds, at least about 1 second, at least about 2 seconds, at least about 5 seconds, at most about 30 seconds, at most about 10 seconds, between about 1 second and about 10 seconds, or between about 1 second about 5 seconds.

In some embodiments, exudate from the wound being treated can rehydrate or at least partially rehydrate the sheet or substrate. In other embodiments, perspiration from the patient can be used to hydrate the sheet or substrate. In still other embodiments, blood derivatives or other products used in wound healing can be used to rehydrate the sheet. In other embodiments, combinations of perspiration, exudate, and other products and blood derivatives can be used to hydrate the sheet or substrate. In other embodiments, “tap” water, salt water, or saliva can be used to hydrate the sheet or substrate.

LLEC/LLEF Systems, Devices; Methods of Use

Embodiments disclosed herein include LLEC and LLEF systems that can promote and/or accelerate the muscle recovery process and optimize muscle performance.

Further, embodiments disclosed herein can increase and/or direct cell migration.

Further embodiments can increase cellular protein sulfhydryl levels and cellular glucose uptake. Increased glucose uptake can result in greater mitochondrial activity and thus increased glucose utilization.

Disclosed methods of use comprise application of a system or device described herein to a tissue, for example skin (such as around the eyes), a joint, a muscle, or a muscle group. In embodiments, the application can be performed prior to, during, or after use of the muscle or muscle group to be treated. For example, a shoulder can be treated prior to engaging in an athletic activity, for example pitching a baseball. Disclosed embodiments can increase glucose uptake, drive redox signaling, increase H₂O₂ production, increase cellular protein sulfhydryl levels, and increase (IGF)-1 R phosphorylation.

Aspects of the invention include devices and methods for increasing capillary density.

Further aspects include a method of directing cell migration using a device disclosed herein. These aspects include methods of improving re-epithelialization.

Further aspects include methods of increasing glucose uptake as well as methods of increasing cellular thiol levels. Additional aspects include a method of energizing mitochondria.

Further aspects include a method of stimulating cellular protein expression.

Further aspects include a method of stimulating cellular DNA synthesis.

Further aspects include a method of stimulating cellular Ca²⁺ uptake.

Embodiments include devices and methods for increasing transcutaneous partial pressure of oxygen. Further embodiments include methods and devices for treating or preventing pressure ulcers.

In embodiments, these systems, devices, and methods can increase ATP production, and angiogenesis, thus accelerating the healing process. Disclosed systems, devices, and methods can also reduce bacterial population and/or proliferation, for example, in and around injuries or wounds. The system, devices, and methods can also increase cellular glucose uptake, thus increasing availability of cellular energy and athletic performance.

Additional aspects include methods of preventing bacterial biofilm formation. Aspects also include a method of reducing microbial or bacterial proliferation, killing microbes or bacteria, killing bacteria through a biofilm layer, or preventing the formation of a biofilm. Embodiments include methods using devices disclosed herein in combination with antibiotics for reducing microbial or bacterial proliferation, killing microbes or bacteria, killing bacteria through a biofilm layer, or preventing the formation of a biofilm.

Further aspects include methods of treating diseases related to metabolic deficiencies, such as diabetes, or other diseases wherein the patient exhibits a compromised metabolic status.

Disclosed embodiments can produce an electrical stimulus and/or can electro-motivate, electro-conduct, electro-induct, electro-transport, and/or electrophorese one or more therapeutic materials in areas of target tissue (e.g., iontophoresis), and/or can cause one or more biologic or other materials in proximity to, on or within target tissue to be rejuvenated. Further disclosure relating to materials that can produce an electrical stimulus can be found in U.S. Pat. No. 7,662,176 entitled FOOTWEAR APPARATUS AND METHODS OF MANUFACTURE AND USE issued Feb. 16, 2010, which is incorporated herein by reference in its entirety.

Methods disclosed herein can include applying a disclosed embodiment to an area to be treated. Embodiments can include selecting or identifying a patient in need of treatment. In embodiments, methods disclosed herein can include application of a device disclosed herein to an area to be treated.

In embodiments, disclosed methods include application to the treatment area or the device of an antibacterial. In embodiments the antibacterial can be, for example, alcohols, aldehydes, halogen-releasing compounds, peroxides, anilides, biguanides, bisphenols, halophenols, heavy metals, phenols and cresols, quaternary ammonium compounds, and the like. In embodiments the antibacterial agent can comprise, for example, ethanol, isopropanol, glutaraldehyde, formaldehyde, chlorine compounds, iodine compounds, hydrogen peroxide, ozone, peracetic acid, formaldehyde, ethylene oxide, triclocarban, chlorhexidine, alexidine, polymeric biguanides, triclosan, hexachlorophene, PCMX (p-chloro-m-xylenol), silver compounds, mercury compounds, phenol, cresol, cetrimide, benzalkonium chloride, cetylpyridinium chloride, ceftolozane/tazobactam, ceftazidime/avibactam, ceftaroline/avibactam, imipenem/MK-7655, plazomicin, eravacycline, brilacidin, and the like.

In embodiments, compounds that modify resistance to common antibacterials can be employed. For example, some resistance-modifying agents may inhibit multidrug resistance mechanisms, such as drug efflux from the cell, thus increasing the susceptibility of bacteria to an antibacterial. In embodiments, these compounds can include Phe-Arg-β-naphthylamide, or β-lactamase inhibitors such as clavulanic acid and sulbactam.

In embodiments, the system can also be used for preventative treatment of tissue injuries. Preventative treatment can include preventing the reoccurrence of previous muscle injuries. For example, a garment can be shaped to fit a patient's shoulder to prevent recurrence of a deltoid injury.

### EXAMPLES

The following non-limiting examples are provided for illustrative purposes only in order to facilitate a more complete understanding of representative embodiments. These examples should not be construed to limit any of the embodiments described in the present specification.

### Example 1

Substrate sheets of a disclosed embodiment (Vomaris Innovations, Inc. Temple Ariz.) were coated with a thin coating of ENERGEL® hydrogel. These coated sheets were then dried for three days at room temperature. The dehydrated hydrogel/sheet was easy to handle, produced no voltage, and would have a long shelf life. When the dehydrated hydrogel/PROCELLERA® sheet was exposed to water, the rehydrated sheet was very much like the original hydrogel coated PROCELLERA® sheet and the original voltage was produced.

### Example 2

A bioelectric device is supplied comprising a multi-array matrix of biocompatible microcells attached to a base sheet and coated with 20 g/ft² of a hydrogel. The sterile device is supplied in a dehydrated state with a peel-able protective layer over the active surface of the device. The device has been shelved for about 6 months.

The user is treating a sutured wound on her arm. She peels the protective layer away from the device, applies a conductive hydrogel to the treatment area, and places the active surface of the device directly on the wound. The device provides a therapeutic electric current to the wound. The wound is protected from infection and heals 50% faster than if covered with a regular bandage.

### Example 3

A bioelectric device is supplied comprising a multi-array matrix of biocompatible microcells attached to a base sheet and coated with 30 g/ft² of an amorphous hydrogel. The sterile device is supplied in a dehydrated state with a peel-able protective layer over the active surface of the device. The device has been shelved for about 3 years.

The user is treating a burn on his back. His caregiver peels the layer away from the device, sprays the device with a misting of water, and places the active surface of the device directly onto the burn site. The device rehydrates with the misting of water and provides a therapeutic electric current to the site. The burn is protected from infection and heals 50% faster than if covered with a regular bandage.

### Example 4

A bioelectric device is supplied comprising a multi-array matrix of biocompatible microcells attached to a base sheet and coated with 50 g/ft² of a hydrated hydrogel sheet. The sterile device is supplied in a dehydrated state with a peel-able layer over the active surface of the device. The device has been shelved for about 2 months.

The user is treating wrinkles on her face. She applies a conductive cream containing epidermal growth factor to the area to be treated, peels the layer away from the device, and places the active surface of the device directly on the treatment site. The device rehydrates with the conductive cream and provides a therapeutic electric current to the site. The treatment is repeated nightly. The wrinkles are noticeably reduced after two weeks of treatment.

### Example 5

A bioelectric device is supplied comprising a multi-array matrix of biocompatible microcells attached to a base sheet and coated with 25 g/ft² of an amorphous hydrogel. The sterile device is supplied in a dehydrated state with a peel-able protective layer over the active surface of the device. The device has been shelved for about 12 months.

The user is treating dry skin on her elbows. She applies a conductive moisturizer cream to the area to be treated, peals the layer away from the device, and places the active surface of the device directly on the treatment site. The device rehydrates with the conductive cream and provides a therapeutic electric current to the site. The treatment is repeated nightly. The dry skin is noticeably reduced after three weeks of treatment.

### Example 6

A bioelectric device is supplied comprising a multi-array matrix of biocompatible microcells attached to a base sheet and coated with 10 g/ft² of a hydrated hydrogel sheet. The sterile device is supplied in a dehydrated state with a peal-able protective layer over the active surface of the device. The device has been shelved for about 4 months. The user is treating dark spots below her eyes. She applies a conductive cream containing keratinocyte growth factor to the area to be treated, peels the layer away from the device, and places the active surface of the device directly on the treatment site. The device rehydrates with the conductive cream and provides a therapeutic electric current to the site. The treatment is repeated nightly. The dark spots are noticeably reduced after three days of treatment.

### Example 7

A bioelectric device is supplied comprising a multi-array matrix of biocompatible microcells attached to a base sheet and coated with 75 g/ft² of a hydrogel. The sterile device is supplied as a single contact layer in a dehydrated state. The device has been shelved for about 4 years.

The user is treating a burn on his back. He sprays the device with a misting of water, and places the active surface of the device directly on the treatment site. The device rehydrates with the misting of water and for a week provides a therapeutic electric current to the site. The burn is protected from infection and heals 70% faster than if covered with a regular bandage.

### Example 8

A bioelectric device is supplied comprising a base sheet coated with 25 g/ft² of a hydrogel comprising a multi-array matrix of biocompatible microcells. The sterile device is supplied in a dehydrated state with a peel-able protective layer over the active surface of the device. The device has been shelved for about 18 months.

The user is treating a laceration to his leg caused by a climbing accident. He wets the device with water from a hydration bladder, peels the protective layer away from the device, and places the active surface of the device directly on the water-misted site. The device rehydrates with the misting of water on the site and for a week provides a therapeutic electric current to the site. The laceration is protected from infection and heals 40% faster than if covered with a regular bandage.

Certain embodiments are described herein, including the best mode known to the inventor for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A tissue treatment system comprising:
a dehydrated composition including an array of microcells and a hydrogel, wherein the dehydrated composition is associated with a substrate.

2. The tissue treatment system of claim 1, wherein the hydrogel comprises a coating of hydrogel on a substrate.

3. The tissue treatment system of claim 2, wherein the hydrated hydrogel is present at about 50 g hydrogel per square foot of the tissue treatment device.

4. The tissue treatment system of claim 1, wherein the substrate includes an adhesive.

5. The tissue treatment system of claim 1, wherein said treatment comprises treatment of skin wrinkles.

6. The tissue treatment system of claim 5, wherein said wrinkles comprise wrinkles around the eye.

7. The tissue treatment system of claim 1, wherein said treatment comprises treatment of a wound.

8. The tissue treatment system of claim 7, wherein said wound comprises a surgical incision.

9. The tissue treatment system of claim 1, wherein the low level micro-current is between 1 and about 200 micro-amperes.

10. A method of treating tissue, the method comprising:
hydrating a composition including an array of microcells and a dehydrated hydrogel thereby proving a hydrated composition, and
applying the hydrated composition to the tissue,
wherein the hydrated composition provides a low level micro-current of 1 and about 400 micro-amperes to the tissue.

11. The method of claim 9, wherein said hydrating comprises applying a liquid to the array of microcells.

12. The method of claim 10 wherein said liquid is a conductive liquid.

13. The method of claim 11 wherein said conductive liquid comprises an active agent.

14. The method of claim 12 wherein said active agent comprises a growth factor.
